(19) United States
(12) Patent Application Publication
(10) Pub. No.: US 2010/0057074 A1
(43) Pub. Date: Mar. 4, 2010

(54) Irrigated Ablation Catheter System and Methods

(76) Inventors: Ricardo D. Roman, San Diego, CA (US); Randell L. Werneth, San Diego, CA (US); Sadaf Soleymani, Reseda, CA (US); Alexander J. Asconeguy, Murrieta, CA (US); Guillermo W. Moratorio, Cardiff by the Sea, CA (US)

Correspondence Address: MEDTRONIC, INC., 710 MEDTRONIC PARKWAY NE, MINNEAPOLIS, MN, 55432-9924, US

(21) Appl. No.: 12/552,617

(22) Filed: Sep. 2, 2009

Related U.S. Application Data
(60) Provisional application No. 61/093,687, filed on Sep. 2, 2008.

(51) Int. Cl. A61B 18/14; A61B 18/18

(52) U.S. Cl. 606/33; 606/41

(57) ABSTRACT

An ablation catheter for performing tissue ablation has an elongate shaft with a lumen. A tip ablation electrode is mounted on the distal end of the shaft. The tip electrode has walls that, together with the plug, define a chamber. The tip electrode has a fluid exit port. The catheter has a cooling fluid delivery system with a connection to a fluid source and a fluid delivery tube within the lumen of the elongate shaft. The fluid delivery tube penetrating said plug and delivers fluid to the fluid exit port. The catheter has a valve assembly in located in the chamber between the fluid delivery tube and the fluid exit port. The valve assembly prevents the flow of cooling fluid through the fluid delivery tube when in the closed position.

### CROSS-REFERENCE TO RELATED APPLICATION

This application claims the benefit of U.S. Provisional Application No. 61/093,687 filed on Sep. 2, 2008.

### DESCRIPTION OF THE INVENTION

1. Field of the Invention

The present invention relates generally to ablation systems and methods for performing targeted tissue ablation in a patient. In particular, the present invention provides catheters which deliver radiofrequency (RF) energy that create safe, precision lesions in tissue such as linear lesions created in cardiac tissue.

2. Background of the Invention

Tissue ablation is used in numerous medical procedures to treat a patient. Ablation can be performed to remove undesired tissue such as cancer cells. Ablation procedures may also involve the modification of the tissue without removal, such as to stop electrical propagation through the tissue in patients with an arrhythmia condition. Often the ablation is performed by passing energy, such as electrical energy, through one or more electrodes and causing the tissue in contact with the electrodes to heat up to an ablative temperature. Ablation procedures can be performed on patients with atrial fibrillation (AF) by ablating tissue in the heart.

Mammalian organ function typically occurs through the transmission of electrical impulses from one tissue area to another. A disturbance of such electrical transmission may lead to organ malfunction. One particular area where electrical impulse transmission is critical for proper organ function is in the heart. Normal sinus rhythm of the heart begins with the sinus node generating an electrical impulse that is propagated uniformly across the right and left atria to the atrioventricular node. Atrial contraction leads to the pumping of blood into the ventricles in a manner synchronous with the pulse.

Atrial fibrillation refers to a type of cardiac arrhythmia where there is disorganized electrical conduction in the atria causing rapid uncoordinated atrial contractions that result in ineffective pumping of blood into the ventricle as well as a lack of synchrony. During AF, the atrioventricular node receives electrical impulses from numerous locations throughout the atria instead of only from the sinus node. These aberrant signals overwhelm the atrioventricular node, producing an irregular and rapid heartbeat. As a result, blood may pool in the atria, increasing the likelihood of blood clot formation. The major risk factors for AF include age, coronary artery disease, rheumatic heart disease, hypertension, diabetes, and thyrotoxicosis. AF affects 7% of the population over age 65.

Atrial fibrillation treatment options are limited. Lifestyle changes only assist individuals with lifestyle related AF. Medication therapy manages AF symptoms, often presents side effects more dangerous than AF, and fails to cure AF. Electrical cardioversion attempts to restore a normal sinus rhythm, but has a high AF recurrence rate. In addition, if there is a blood clot in the atria, cardioversion may cause the clot to leave the heart and travel to the brain (causing a stroke) or to some other part of the body. What are needed are new methods for treating AF and other medical conditions involving disorganized electrical conduction.

Various ablation techniques have been proposed to treat AF, including the Cox-Maze ablation procedure, linear ablation of various regions of the atrium, and circumferential ablation of pulmonary vein ostia. The Cox-Maze ablation procedure and linear ablation procedures are tedious and time-consuming, taking several hours to accomplish. Current pulmonary vein ostial ablation is proving to be difficult to do, and has lead to rapid stenosis and potential occlusion of the pulmonary veins. All ablation procedures involve the risk of inadvertently damaging untargeted tissue, such as the esophagus while ablating tissue in the left atrium of the heart. There is therefore a need for improved atrial ablation products and techniques that create efficacious lesions in a safe manner.

### SUMMARY OF THE INVENTION

Several unique ablation catheters and ablation catheter systems and methods are provided which map and ablate surface areas within the heart chambers of a patient, with one or few catheter placements. Any electrocardiogram signal site (e.g. a site with aberrant signals) or combination of multiple sites that are discovered with this placement may be ablated. In alternative embodiments, the ablation catheters and systems may be used to treat non-cardiac patient tissue, such as tumor tissue.

According to a first aspect of the invention, an ablation catheter for performing a medical procedure on a patient is provided. The ablation catheter comprises an elongate shaft with a proximal portion including a proximal end and a distal end, and a distal portion with a proximal end and a distal end. The elongate shaft further comprises a shaft ablation assembly and a distal ablation assembly configured to deliver energy, such as RF energy, to tissue. The shaft ablation assembly is proximal to the distal end of the distal portion, and includes at least one shaft ablation element fixedly attached to the shaft and configured to deliver ablation energy to tissue. The distal ablation assembly is at the distal end of the distal portion and includes at least one tip ablation element configured to deliver ablation energy to tissue.

The ablation elements of the present invention can deliver one or more forms of energy, preferably RF energy. The ablation elements may have similar or dissimilar construction, and may be constructed in various sizes and geometries. The ablation elements may include one or more thermocouples, such as two thermocouples mounted 180° from each other on an ablation element inner or outer surface. The ablation elements may include means of dissipating heat, such as increased surface area of projecting fins. The ablation elements may have asymmetric geometries, such as electrodes with thin and thick walls positioned on the inside and/or outside of one or more curved deflection geometries. In one embodiment, one or more ablation elements is configured in a tubular geometry, and the wall thickness to outer diameter approximates a 1:10 ratio. In another embodiment, one or more ablation elements is configured to record, or map electrical activity in tissue such as mapping of cardiac electrograms. In yet another embodiment, one or more ablation elements is configured to deliver pacing energy, such as to energy delivered to pace the heart of a patient.

The ablation catheters of the present invention may be used to treat one or more medical conditions by delivering ablation energy to tissue. Conditions include an arrhythmia of the heart, cancer, and other conditions in which removing or denaturing tissue improves the patient's health.

According to another aspect of the invention, a method of treating proximal or chronic atrial fibrillation is provided. An ablation catheter of the present invention may be placed in the coronary sinus of the patient, such as to map electrograms and/or ablate tissue, and subsequently placed in the left or right atrium to ablate tissue. The ablation catheter may be placed to ablate one or more tissue locations including but not limited to: fasicals around a pulmonary vein; and the mitral isthmus.

According to another aspect of the invention, a method of treating atrial flutter is provided. An ablation catheter of the present invention may be used to achieve bi-directional block, such as by placement in one or more locations in the right atrium of the heart.

According to another aspect of the invention, a method of ablating tissue in the right atrium of the heart is provided. An ablation catheter of the present invention may be used to: create lesions between the superior vena cava and the inferior vena cava; the coronary sinus and the inferior vena cava; the superior vena cava and the coronary sinus; and combinations of these. The catheter can be used to map and/or ablate the sinus node, such as to treat sinus node tachycardia.

According to another aspect of the invention, a method of treating ventricular tachycardia is provided. An ablation catheter of the present invention may be placed in the left or right ventricles of the heart, induce ventricular tachycardia by delivering pacing energy, and ablating tissue to treat the patient.

According to another aspect of the invention, an ablation catheter with an irrigated tip is provided. In one embodiment, a fluid delivery system delivers cooling fluid to a distal exit port in fluid communication with a fluid exit channel. In another aspect, the distal end of the ablation catheter has walls that define a chamber. A fluid delivery system causes cooling fluid to flow into the chamber. In another aspect of the invention, a valve is installed in the chamber. The valve moves between an open position, in which irrigation fluid is delivered to the distal exit port. In a closed position, the valve prevents irrigation fluid from flowing to the distal exit port. The valve may be controlled in response to a temperature measurement, or may be controlled based on the timing of the delivery of energy to the ablation elements.

According to another aspect of the invention, an ablation catheter has an elongate shaft to which are attached shaft ablation electrodes and a tip ablation element having a tip electrode. Both the shaft ablation electrodes and the tip ablation element have fluid exit ports. A fluid delivery system delivers fluid to the shaft ablation electrode fluid exit ports and to the tip ablation element fluid exit port.

### BRIEF DESCRIPTION OF THE DRAWINGS

The accompanying drawings, which are incorporated in and constitute a part of this specification, illustrate various embodiments of the present invention, and, together with the description, serve to explain the principles of the invention. In the drawings:

FIG. 1 illustrates a side view of an ablation catheter, consistent with the present invention.

FIG. 1A illustrates a schematic view of an ablation system, consistent with the present invention.

FIG. 2 illustrates an anatomical view of an ablation catheter placed into the left atrium of a heart, consistent with the present invention.

FIG. 3A illustrates a side view of the distal portion of a shaft of an ablation catheter, with a staircase joint, consistent with the present invention.

FIG. 3B illustrates a side, partial sectional view of the shaft of FIG. 3A.

FIG. 3C illustrates a side view of an alternative tapered joint, consistent with the present invention.

FIG. 3D illustrates a side view of an alternative toothed joint, consistent with the present invention.

FIG. 4 illustrates a side view of an ablation catheter, consistent with the present invention.

FIG. 4A illustrates a side view of the distal end of the ablation catheter of FIG. 4.

FIG. 4B illustrates a cross sectional view of the shaft of the ablation catheter of FIG. 4.

FIG. 4C illustrates a side view of a shaft subassembly of the catheter of FIG. 4.

FIG. 4D illustrates a side sectional view of a portion of the shaft subassembly of FIG. 4C.

FIG. 4E illustrates a cross sectional view of the catheter shaft subassembly of FIG. 4C.

FIGS. 5 and 5A illustrate a schematic view of an ablation system including a fluid delivery system and cooled ablation catheter, consistent with the present invention.

FIGS. 6A, 6B and 6C illustrate multiple views of a tip electrode including a slit for passage of cooling fluid, consistent with the present invention.

FIGS. 7A and 7B illustrate methods of applying the ablation catheter of FIGS. 28-C to tissue, consistent with the present invention.

FIGS. 8A and 8B illustrate two tip electrodes with exit ports for passage of cooling fluid, consistent with the present invention.

FIGS. 8C and 8D illustrate side and end views, respectively, of a tip electrode with a spiral channel for passage of cooling fluid, consistent with the present invention.

FIGS. 8E and 8F illustrate side and end views, respectively, of a tip electrode including multiple internal fins, consistent with the present invention.

FIGS. 9A, 9B and 9C illustrate multiple views of a band electrode including multiple holes for passage of cooling fluid, consistent with the present invention.

FIG. 10 illustrates a side sectional view of a pair of electrodes mounted to an ablation catheter shaft, the electrodes including portions which reside within a lumen of the shaft, consistent with the present invention.

FIG. 11 illustrates a perspective view of an ablation catheter with cooling means, consistent with the present invention.

FIG. 12 illustrates a perspective, partial sectional view of the distal end of an ablation catheter with cooling means, consistent with the present invention.

FIGS. 13A and 13B illustrate end and side views of the distal portion of an ablation catheter with cooling means, consistent with the present invention.

FIG. 14 illustrates a side view of the distal portion of an ablation catheter with cooling means, consistent with the present invention.

FIG. 15 illustrates a perspective view of an ablation catheter with cooling means, consistent with the present invention.

FIGS. 16A and 16B illustrate sectional side views of the distal portion of an ablation catheter including a temperature controlled valve, consistent with the present invention.

FIG. 17 illustrates a sectional side view of the distal portion of an ablation catheter with cooling means, consistent with the present invention.

FIG. 18 illustrates a perspective, partial sectional view of a tip electrode with cooling means, consistent with the present invention.

FIGS. 19A and 19B illustrate side and end views, respectively, of a tip electrode with multiple portions, consistent with the present invention.

FIGS. 20A and 20B illustrate side and end views, respectively, of a tip electrode with multiple portions, consistent with the present invention.

### DESCRIPTION OF THE EMBODIMENTS

Reference will now be made in detail to the present embodiments of the invention, examples of which are illustrated in the accompanying drawings. Wherever possible, the same reference numbers will be used throughout the drawings to refer to the same or like parts.

The present invention provides catheters for performing targeted tissue ablation in a subject. In some embodiments, the catheters comprise an elongate shaft having a proximal end and distal end and preferably a lumen extending at least partially therebetween. The catheter is preferably of the type used for performing intracardiac procedures, typically being introduced from the femoral vein in a patient's leg or from a vessel in the patient's neck. The catheter is preferably introducible through a sheath, such as a transeptal sheath, and also preferably has a steerable tip that allows positioning of the distal portion such as when the distal end of the catheter is within a heart chamber. The catheters include ablation elements located at the distal end of the shaft (tip electrodes), as well as ablation elements located on an exterior surface of the shaft proximal to the distal end (shaft electrodes). The tip electrodes may be fixedly attached to the distal end of the shaft, or may be mounted on an advancable and/or expandable carrier assembly. The carrier assembly may be attached to a control shaft that is coaxially disposed and slidingly received within the lumen of the shaft. The carrier assembly is deployable by activating one or more controls on a handle of the catheter, such as to engage one or more ablation elements against cardiac tissue, typically atrial wall tissue or other endocardial tissue. The shaft may include deflection means, such as means operably connected to a control on a handle of the catheter. The deflection means may deflect the distal portion of the shaft in one or more directions, such as deflections with two symmetric geometries, two asymmetric geometries, or combinations of these. Asymmetries may be caused by different radius of curvature, different length of curvature, differences in planarity, other different 2-D shapes, other different 3-D shapes, and the like.

In particular, the present invention provides ablation catheters with multiple electrodes that provide electrical energy, such as radiofrequency (RF) energy, in monopolar (unipolar), bipolar or combined unipolar-bipolar fashion, as well as methods for treating conditions such as paroxysmal atrial fibrillation, chronic atrial fibrillation, atrial flutter, supra ventricular tachycardia, atrial tachycardia, ventricular tachycardia, ventricular fibrillation, and the like, with these devices.

The normal functioning of the heart relies on proper electrical impulse generation and transmission. In certain heart diseases (e.g., atrial fibrillation) proper electrical generation and transmission are disrupted or are otherwise abnormal. In order to prevent improper impulse generation and transmission from causing an undesired condition, the ablation catheters and RF generators of the present invention may be employed.

One current method of treating cardiac arrhythmias is with catheter ablation therapy. Physicians make use of catheters to gain access into interior regions of the body. Catheters with attached electrode arrays or other ablating devices are used to create lesions that disrupt electrical pathways in cardiac tissue. In the treatment of cardiac arrhythmias, a specific area of cardiac tissue having aberrant conductive pathways, such as atrial rotors, emitting or conducting erratic electrical impulses, is initially localized. A user (e.g., a physician) directs a catheter through a main vein or artery into the interior region of the heart that is to be treated. The ablating element (or elements) is next placed near the targeted cardiac tissue that is to be ablated. The physician directs energy, provided by a source external to the patient, from one or more ablation elements to ablate the neighboring tissue and form a lesion. In general, the goal of catheter ablation therapy is to disrupt the electrical pathways in cardiac tissue to stop the emission and/or prevent the propagation of erratic electric impulses, thereby curing the focus of the disorder. For treatment of AF, currently available methods and devices have shown only limited success and/or employ devices that are extremely difficult to use or otherwise impractical.

The ablation systems of the present invention allow the generation of lesions of appropriate size and shape to treat conditions involving disorganized electrical conduction (e.g., AF). The ablation systems of the present invention are also practical in terms of ease-of-use and limiting risk to the patient (such as in creating an efficacious lesion while minimizing damage to untargeted tissue), as well as significantly reducing procedure times. The present invention addresses this need with, for example, arrangements of one or more tip ablation elements and one or more shaft ablation elements configured to create a linear lesion in tissue, such as the endocardial surface of a chamber of the heart, by delivery of energy to tissue or other means. The electrodes of the present invention may include projecting fins or other heat dissipating surfaces to improve cooling properties. The distal portions of the catheter shafts of the present invention may deflect in two or more symmetric or asymmetric geometries, such as asymmetric geometries with different radius of curvature or other geometric shape differences. The ablation catheters and RF generators of the present invention allow a clinician to treat a patient with AF in a procedure much shorter in duration than current AF ablation procedures. The lesions created by the ablation catheters and RF generators of the present invention are suitable for inhibiting the propagation of inappropriate electrical impulses in the heart for prevention of reentrant arrhythmias, while minimizing damage to untargeted tissue, such as the esophagus or phrenic nerve of the patient.

### DEFINITIONS

To facilitate an understanding of the invention, a number of terms are defined below.

As used herein, the terms “subject” and “patient” refer to any animal, such as a mammal like livestock, pets, and preferably a human. Specific examples of “subjects” and “patients” include, but are not limited, to individuals requiring medical assistance, and in particular, requiring AF catheter ablation treatment.

As used herein, the terms “catheter ablation” or “ablation procedures” or “ablation therapy,” and like terms, refer to what is generally known as tissue destruction procedures. Ablation is often used in treating several medical conditions, including abnormal heart rhythms. It can be performed both surgically and non-surgically. Non-surgical ablation is typically performed in a special lab called the electrophysiology (EP) laboratory. During this non-surgical procedure an ablation catheter is inserted into the heart using fluoroscopy for visualization, and then an energy delivery apparatus is used to direct energy to the heart muscle via one or more ablation elements of the ablation catheter. This energy either “disconnects” or “isolates” the pathway of the abnormal rhythm (depending on the type of ablation). It can also be used to disconnect the conductive pathway between the upper chambers (atria) and the lower chambers (ventricles) of the heart. For individuals requiring heart surgery, ablation can be performed during coronary artery bypass or valve surgery.

As used herein, the term “ablation element” refers to an energy delivery element, such as an electrode for delivering electrical energy. Ablation elements can be configured to deliver multiple types of energy, such as ultrasound energy and cryogenic energy, either simultaneously or serially. Electrodes can be constructed of a conductive plate, cylinder or tube, a wire coil, or other means of conducting electrical energy through contacting tissue. In unipolar energy delivery, the energy is conducted from the electrode, through the tissue to a ground pad, such as a conductive pad attached to the back of the patient. The high concentration of energy at the electrode site causes localized tissue ablation. In bipolar energy delivery, the energy is conducted from a first electrode to one or more separate electrodes, relatively local to the first electrode, through the tissue between the associated electrodes. Bipolar energy delivery results in more precise, shallow lesions while unipolar delivery results in deeper lesions. Both unipolar and bipolar deliveries provide advantages, and the combination of their use is a preferred embodiment of this application.

As used herein, the term “return pad” refers to a surface electrode mounted to the patient's body, typically on the patient's back. The return pad receives the RF ablation currents generated during unipolar power delivery. The return pad is sized (large enough) such that the high temperatures generated remain within a few millimeters of the specific ablation catheter's electrode delivering the unipolar power.

As used herein, the term “RF output” refers to an electrical output produced by the RF generator of the present invention. The RF output is electrically connected to a jack or other electro-mechanical connection means which allows electrical connection to one or more ablation elements (e.g. electrodes) of an ablation catheter. The RF output provides the RF energy to the ablation element to ablate tissue with bipolar and/or unipolar energy.

As used herein, the term “channel” refers to a pair of RF outputs between which bipolar energy is delivered. Each of the RF outputs in a channel may also deliver unipolar energy (simultaneous and/or sequential to bipolar energy delivery), such as when a return pad is connected.

As used herein, the term “targeted tissue” refers to tissue to be ablated, as identified by the clinician and/or one or more algorithms (e.g. algorithms of the system or algorithms otherwise available to the clinician). Lesions created in targeted tissue disconnect an aberrant electrical pathway causing an arrhythmia, or treat other undesired tissue such as cancer tissue.

As used herein, the term “untargeted tissue” refers to tissue which is desired to avoid damage by ablation energy, such as the esophagus or phrenic nerve in an arrhythmia ablation procedure.

As used herein, the term “power delivery scheme” refers to a set of ablation parameters to be delivered during a set ablation time, and used to safely create an effective lesion in targeted tissue. Power delivery scheme parameters include but are not limited to: type (bipolar and/or unipolar) of energy delivered; voltage delivered; current delivered; frequency of energy delivery; duty cycle parameter such as duty cycle percentage or length of period; field parameter such as configuration of fields or number of fields in set that repeats; and combinations thereof.

As used herein, the term “proximate” is used to define a particular location, such as “ablating tissue proximate the sinus node”. For the purpose of this application, proximate shall include the area neighboring a target as well as the target itself. For the example above, the tissue receiving the ablation energy would be tissue neighboring the sinus node as well as the sinus node itself.

The present invention provides structures that embody aspects of the ablation catheter. The present invention also provides RF generators for providing ablation energy to the ablation catheters. The illustrated and preferred embodiments discuss these structures and techniques in the context of catheter-based cardiac ablation. These structures, systems, and techniques are well suited for use in the field of cardiac ablation.

However, it should be appreciated that the invention is applicable for use in other tissue ablation applications such as tumor ablation procedures. For example, the various aspects of the invention have application in procedures for ablating tissue in the prostrate, brain, gall bladder, uterus, and other regions of the body, preferably regions with an accessible wall or flat tissue surface, using systems that are not necessarily catheter-based. In some embodiments, the target tissue is tumor tissue.

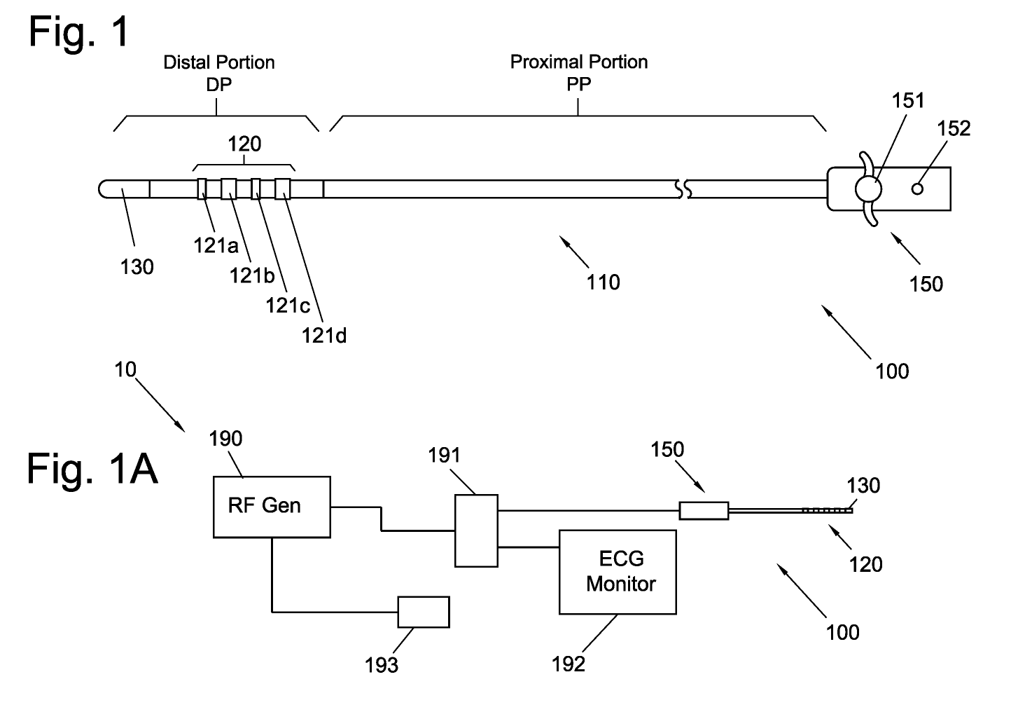

The ablation catheters and systems of the present invention have advantages over previous prior art devices. FIGS. 1-26 show various embodiments of the ablation catheters and systems of the present invention. The present invention is not limited to these particular configurations.

Referring now to FIG. 1, an embodiment of an ablation catheter of the present invention is illustrated. Ablation catheter 100 includes flexible shaft 110 which includes proximal portion PP and distal portion DP. Handle 150 is located on the proximal end of proximal portion PP and includes multiple controls, knob 151 and button 152. Button 152 is configured to initiate and/or discontinue delivery of energy to one or more ablation elements located in distal portion DP. Knob 151 is configured, when rotated, to cause distal portion DP to deflect in one or more directions, such as to curve in one direction when rotated clockwise, and another direction when rotated counter-clockwise. In one embodiment, described in detail herebelow, knob 151 is attached to two steering wires which are captured in the distal portion DP and cause bi-directional steering such as symmetric or asymmetric steering. In alternative embodiments, 1, 3, 4 or more steering wires may be incorporated, such as steering wires separated by 120° or 90°, causing deflection in a single plane, or three or more planes. Each deflection may have a simple geometry such as a single plane, fixed radius curve, or more complex geometries.

Additional controls may be integrated into handle 150 to perform additional functions. A connector, not shown, is integral to handle 150 and allows electrical connection of ablation catheter 100 to one or more separate devices such as an RF generator or other energy delivery unit; a temperature monitoring system, an ECG monitoring system; a cooling source; an inflation source, and/or numerous other electro-mechanical devices.

Distal portion DP includes shaft ablation assembly 120 which includes multiple ablation elements 121a, 121b, 121c and 121d. Distal portion DP further includes distal ablation assembly 130, which preferably includes at least one ablation element, such as an atraumatic (e.g. rounded tip), platinum, tip electrode configured to deliver RF energy to tissue. In a preferred configuration, ablation elements 121a, 121b, 121c and 121d are platinum electrodes configured to deliver unipolar energy (energy delivered between that electrode and a return pad), and/or bipolar energy (energy delivered between that electrode an a second electrode in general proximity to the first electrode). Distal ablation assembly 130 may include multiple ablation elements, such as multiple platinum electrodes separated by an insulator, and/or deployable from the distal end of shaft 110 (e.g. via a control on handle 150). Distal ablation assembly 130 and shaft ablation assembly 120 preferably include one or more temperature sensors, not shown but preferably at least one thermocouple mounted to each ablation element.

In a preferred embodiment, the ablation elements of catheter 100 are electrodes attached to signal wires, not shown but traveling within shaft 110 and electrically connecting to an electrical connector on handle 150. The signal wires, described in detail in reference to subsequent figures, carry power to the electrodes for unipolar and/or bipolar energy delivery, and also receive signals from the electrodes such as ECG mapping signals of the human heart. The signal wires can transmit or receive information from one or more other functional elements of catheter 100, also not shown but preferably a sensor such as a thermocouple or a transducer such as an ultrasound crystal.

In a preferred configuration, two signal wires of approximately 36 gauge are connected to a tip electrode of distal ablation assembly 130. The two 36 gauge wires can each simultaneously deliver unipolar energy to the tip electrode, such as to deliver up to 45 watts of unipolar energy (approximately 45 Watts being a preferred maximum energy delivery for a tip electrode of the present invention). Minimizing of the diameter of the signal wires provides numerous advantages such as minimizing the required diameter of shaft 110 as well as preventing undesired stiffening of shaft 110. In an alternative embodiment, one or both of the 36 gauge wires is configured to prevent embolization of the tip electrode, such as when the joint between the tip electrode and shaft 110 fails. One or both of these signal wires can be attached to a temperature sensor such as a thermocouple and transmit temperature information back to an electrical connector of handle 150.

In a preferred configuration, a signal wire of approximately 36 gauge and a signal wire of approximately 40 gauge are connected to a shaft electrode such as shaft ablation element 121a, 121b, 121c or 121d. Bipolar or unipolar energy can be delivered through the 36 gauge wire, such as a power up to 20 watts (approximately 20 Watts being a preferred maximum energy delivery for a shaft electrode of the present invention). Minimizing of the diameter of the signal wires provides numerous advantages such as minimizing the required diameter of shaft 110 as well as preventing undesired stiffening of shaft 110. One or both of these signal wires can be attached to a temperature sensor such as a thermocouple and transmit temperature information back to an electrical connector of handle 150.

Referring now to FIG. 1A, an embodiment of an ablation system of the present invention is illustrated. System 10 includes ablation catheter 10 and an energy delivery system such as RF generator 190. Ablation catheter 100 is attached to ECG interface 191, which in turn is attached to RF generator 190; preferably an RF generator configured to delivery unipolar and bipolar RF energy to the ablation elements of ablation catheter 100. ECG Interface 191 is also attached to ECG monitor 192 such that the high power energy delivered to ablation catheter 100 by RF generator 190 is isolated from ECG monitor 192. RF generator 190 preferably is connected to a power source (not shown but preferably an electrical outlet connected to 110 or 220 AC volts) and is configured to deliver unipolar and bipolar RF energy to one or more electrodes on distal ablation assembly 130 and shaft ablation assembly 120. RF generator 190 is also attached to a return pad, patient return electrode 193, configured to receive the unipolar energy delivered to one or more ablation elements of catheter 100.

Alternatively or additionally, RF generator 190 may deliver other forms of energy, including but not limited to: acoustic energy and ultrasound energy; electromagnetic energy such as electrical, magnetic, microwave and radiofrequency energies; thermal energy such as heat and cryogenic energies; chemical energy; light energy such as infrared and visible light energies; mechanical energy; radiation; and combinations thereof.

In a preferred embodiment, RF generator 190 provides ablation energy to one or more ablation elements of catheter 100 by sending power to one or more independently controlled RF outputs of RF generator 190. The independent control of each RF output allows a unique, programmable power delivery signal to be sent to each electrode of ablation catheter 100. The independent control of each RF output further allows unique (independent) closed loop power delivery, such as power delivery regulated by tissue temperature (e.g. regulated to tissue temperature of 60° C.) information received from one or more temperature sensors integral to the attached ablation catheter and/or from sensors included in a separate device.

The number of RF outputs can vary as required by the design of the attached ablation catheter. In a preferred embodiment, four to twelve independent RF outputs are provided, such as when the system of the present invention includes a kit of ablation catheters including at least one catheter with from four to twelve electrodes. In another preferred embodiment, sixteen or more independent RF outputs are provided, such as when the system of the present invention includes a kit of ablation catheters including at least one catheter with sixteen or more electrodes.

Unipolar delivery is accomplished by delivering currents that travel from an RF output of RF generator 190 to an electrically attached electrode of ablation catheter 100, through tissue to return pad 193, and back to RF generator 190 to which return pad 193 has been connected. Bipolar delivery is accomplished by delivering current between a first RF output which has been electrically connected to a first electrode of an ablation catheter and a second RF output which has been electrically connected to a second electrode of the ablation catheter, the current traveling through the tissue between and proximate the first and second electrodes. Combo mode energy delivery is accomplished by combining the unipolar and bipolar currents described immediately hereabove. The user (e.g. a clinician or clinician's assistant) may select or deselect RF outputs receiving energy to customize therapeutic delivery to an individual patient's needs.

In another preferred embodiment, five different pre-set energy delivery options are provided to the user: unipolar-only, bipolar-only, and 4:1, 2:1 and 1:1 bipolar/unipolar ratios. The ratios refer to the relative amount of power delivered by each mode of power. A bipolar-only option provides the shallowest depth lesion, followed by 4:1, then 2:1, then 1:1 and then unipolar-only which provides the deepest depth lesion. The ability to precisely control lesion depth increases the safety of the system and increases procedure success rates as target tissue can be ablated near or over important structures. In an alternative embodiment, currents are delivered in either unipolar mode or a combination mode consisting of bipolar and unipolar energy. The preferred embodiment, which avoids the use of bipolar-only energy, has been shown to provide numerous benefits including reduction of electrical noise generated by switching off the return pad circuit (e.g. to create bipolar-only mode).

In another preferred embodiment, RF generator 190 includes multiple independent PID control loops that utilize measured tissue temperature information to regulate (i.e. provide closed loop) energy delivered to an ablation catheter's electrodes. In one embodiment, RF generator 190 includes twelve separate, electrically-isolated temperature sensor inputs. Each temperature input is configured to receive temperature information such as from a sensor such as a thermocouple. The number of temperature inputs can vary as required by the design. In a preferred embodiment, four to twelve independent inputs are provided, such as when the system of the present invention includes a kit of ablation catheters including at least one catheter with from four to twelve thermocouples. In another preferred embodiment, sixteen or more independent temperature inputs are provided, such as when the system of the present invention includes a kit of ablation catheters including at least one catheter with sixteen or more thermocouples.

Ablation target temperatures are user-selectable and automatically achieved and maintained throughout lesion creation, regardless of blood flow conditions and/or electrode contact scenarios. Temperature target information is entered via a user interface of RF generator 190. The user interface is configured to allow an operator to input system parameter information including but not limited to: electrode selection; power delivery settings, targets and other power delivery parameters; and other information. The user interface is further configured to provide information to the operator, such as visual and audible information including but not limited to: electrode selection, power delivery parameters and other information. Automatic temperature-controlled lesion creation provides safety and consistency in lesion formation. Typical target temperature values made available to the operator range from 50 to 70° C.

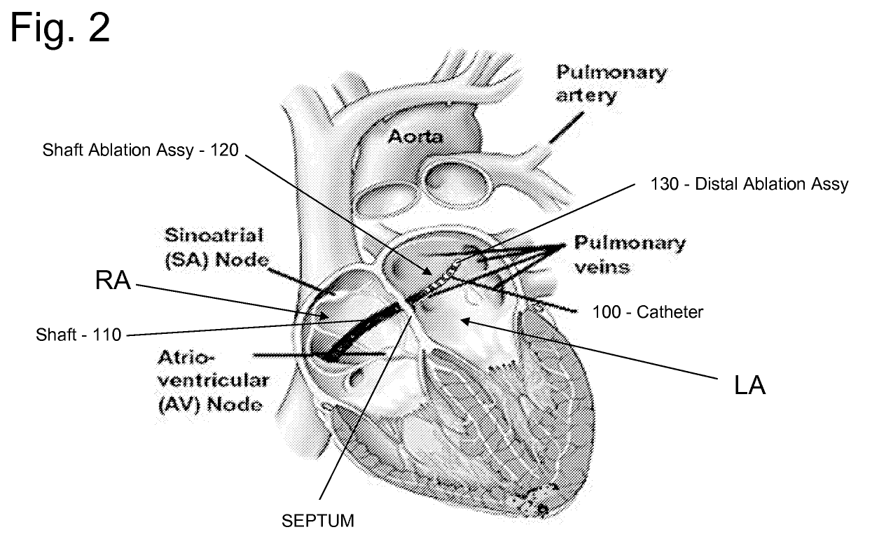

Referring now to FIG. 2, a preferred method of the present invention is illustrated. For the purposes of FIG. 2, it is generally noted that all designs shown may include multiple electrodes, and in preferred configurations also includes a return pad (a large surface area electrode often attached to the patient's back). At least one pair of electrodes, and often many pairs, may be activated or powered with appropriately-powered potential differences to create RF waves that penetrate and ablate desired tissue. If the powering occurs between a pair of electrodes, it is termed “bipolar”. If the powering occurs between one electrode and the return pad, it is termed “unipolar”. If both bipolar and unipolar power is delivered simultaneously to tissue, it is termed “combo” or “combo mode”.

The cross-section of the human heart depicts the atrioventricular node and the sinoatrial node of the right atrium RA, the pulmonary vein ostia of the left atrium LA, and the septum with the right atrium RA and the left atrium LA. Catheter 100 is shown entering the right atrium RA, passing through the septum, and terminating in left atrium LA. The distal portion of shaft 110 includes shaft ablation assembly 120 and distal ablation assembly 130 as shown in FIG. 2. Distal ablation assembly 130 preferably includes a platinum electrode at its tip. Shaft ablation assembly 120 preferably includes two to six (or more) platinum electrodes secured to the outer diameter of shaft 110. The electrodes of distal ablation assembly 130 and shaft ablation assembly 120 may be configured to deliver unipolar and bipolar RF energy to the heart tissue, such as the tissue of the left atrium LA.

Catheter 100, provided in a sterile form such as via e-beam sterilization and sterile packaging, may be percutaneously inserted in either femoral vein, advanced toward the heart through the inferior vena cava (IVC), and into the right atrium. Through the use of a previously placed transeptal sheath (e.g. a deflectable or fixed shape 9.5 Fr sheath), catheter 100 may be advanced through the septum into the left atrium LA to perform a left atrial ablation. In an alternative embodiment, catheter 100 may be advanced only into the right atrium RA to perform an ablation procedure in the right atrium RA or coronary sinus.

In a preferred method, ablation catheter 100 is configured to treat paroxysmal atrial ablation and/or chronic atrial ablation. In these procedures, catheter 100 can be used as a reference catheter (configured to map electrical activity) in the coronary sinus. Alternatively or additionally, ablation catheter 100 may perform an ablation in the right atrium RA or left atrium LA, such as an ablation of: the fasicals proximate the pulmonary veins; the mitral isthmus; and other right atrial RA and left atrial LA locations. In another preferred embodiment, ablation catheter 100 is configured to be transformed into multiple deflection geometries such that the left and/or right atria can be treated utilizing one or more of these multiple deflection geometries. In a preferred method, a first deflection radius (e.g. a radius less than or equal to 28 mm) is used to ablate tissue on the “roof” of the left atrium, in tissue proximate the septum and/or tissue close to the posterior wall. A second deflection radius, larger than the first deflection radius (e.g. greater than or equal to 28 mm), is used to ablate the floor of the left atrium. In another preferred method, a small deflection radius is used to treat atria with a relatively small volume, and a larger deflection radius is used to treat larger atria (e.g. an enlarged atria of a chronic AF patient). In yet another preferred method, an ablation catheter with a first deflection geometry is configured to treat the right atrium, the ablation catheter further configured with a second deflection geometry, different than the first deflection geometry and configured to treat the left atrium. Differences in deflection geometry may include different radius of curvature, such as a first radius of curvature less than or equal to 28 mm and a second radius of curvature greater than or equal to 28 mm.

Ablation catheter 100 may include a handle with a rotating knob. The rotating knob may be operably connected to one or more steering wires such that rotation of the knob in a first direction causes the first radius to be generated and rotating the knob in an opposite direction causes the second radius to be generated.

In another preferred method, ablation catheter 100 may be used to treat atrial flutter. The ablation procedure may be completed with as little as one or two catheter placements allowing the operator to block the aberrant signals causing the flutter. In a preferred method, ablation catheter 100 blocks the aberrant signals with less than 5 placements, preferably less than 3 placements. In another preferred method, the ablation procedure results in bi-directional block. Ablation catheter 100 may be used to treat atrial flutter by creating a lesion along the length of the isthmus, such as with a single ablation. Alternatively or additionally, a lesion may be created proximate the tricuspid annulus, a location known to often include aberrant electrical signals associated with atrial flutter. In another preferred embodiment, ablation catheter 100 includes a deflectable portion which can be deflected in a first direction with a first radius of curvature, and in a second direction with a second, larger radius of curvature. The smaller first radius of curvature is used to ablate the concave portion of the isthmus, and the larger second radius of curvature is used to create one or more lesions in the tissue proximate the tricuspid annulus. In a preferred embodiment, the smaller radius of curvature is at or below 28 mm and the larger radius of curvature is at or above 28 mm.

Alternatively or additionally, ablation catheter 100 may be used in other methods to treat atrial flutter. In a preferred embodiment, in a first step, the distal portion of ablation catheter 100 is placed relatively perpendicular to the isthmus, such as with the middle portion of the shaft ablation assembly at a point along the isthmus; in a second step pacing energy is applied by one or more tip ablation elements while electrograms are recorded by one or more shaft ablation elements; and in a third step pacing energy is applied by one or more shaft ablation elements while electrograms are recorded by one or more tip ablation elements. Steps 2 and 3 may be repeated until desired electrograms are recorded. In an alternative embodiment, step 3 is performed before step 2. Alternatively or additionally, shaft ablation assembly 120 includes multiple ablation elements, such as multiple electrodes configured to both deliver RF energy and record electrograms. One electrode is most proximate the proximal end of ablation catheter 100, and one or more electrodes (“middle electrodes”) are located between this most proximate electrode and the distal ablation assembly 130. These one or more middle electrodes can be used to measure “split potential” electrograms, such as electrograms used to confirm adequate block has been achieved. These middle electrodes can be used to identify tissue needing further ablation.

Alternatively or additionally, ablation catheter 100 may be used in yet other methods to treat atrial flutter. In a preferred embodiment, in a first step, the distal portion of ablation catheter 100 is deflected 90° or more, such as a deflection of 135° or more (deflections not shown). The one or more ablation elements of shaft ablation assembly 120 and/or distal ablation assembly 130 can be used to deliver ablation energy to tissue proximate the eustachian ridge and/or valley. In one embodiment, ablation catheter 100 includes a deflection mechanism (as described in various embodiments herebelow), and the 90° or more deflection is accomplished by an operator activating the deflection mechanism, such as via a control on a handle of ablation catheter 100 (handle and control not shown but described in detail in reference to various embodiments herebelow). Alternatively or additionally, the 90° or more deflection can be accomplished by pressing the distal portion of ablation catheter 100 against tissue, such as tissue proximate the eustachian ridge and/or valley.

Ablation catheter 100 may be used in various ablation procedures in the right atrium RA of the heart. In a preferred method, a lesion is created between one or more of: the superior vena cava (SVC) and the inferior vena cava (IVC); the coronary sinus (CS) and the IVC; and the SVC and the IVC. In one embodiment, a lesion is created between all three locations described immediately hereabove. In another preferred right atrial method, ablation catheter 100 is used to treat sinus node tachycardia by measuring electrograms in tissue proximate the sinus node and ablating tissue proximate the sinus node.

Ablation catheter 100 may be used to ablate tissue proximate or within the coronary sinus (CS). In a preferred method, ablation catheter 100 delivers bipolar RD energy, such as to improve the treatment of atrial fibrillation (e.g. improving acute and/or chronic results of AF therapy).

Ablation catheter 100 may be used to treat ventricular tachycardia. In a preferred method, the distal portion of ablation catheter 100 is placed in the right or left ventricle, and pacing energy is delivered by one or more ablation elements, such as electrodes, inducing ventricle tachycardia. Information received or determined by the pacing step, is used by an operator to deliver ablation energy to the ventricle with one or more ablation elements of ablation catheter 100. The information may be used to selectively ablate tissue, such as to determine ablation location(s), ablation settings, or another ablation parameter.

The ablation catheter 100 of the present invention is preferably configured to create linear lesions in tissue of a patient, such as heart tissue. The catheter may be further configured to ablate tissue in an arrhythmia treating procedure such as a procedure to treat AF. Ablation catheter may be used in combination with other ablation catheters, such as catheters configured to be used prior to ablation catheter 100 and/or catheters configured to create longer or otherwise larger lesions in tissue such as the left atrium LA. In this subsequent use, ablation catheter 100 may be configured to create smaller lesions that complete a set of lesions to treat AF. These smaller lesions are often referred to as “touch up” lesions.

Ablation catheter 100 and the other ablation catheters of the present invention may be configured to ablate tissue and also map electrical activity in tissue, such as intracardiac electrogram activity. Mapping of AF in humans has shown that areas of complex fractionated atrial electrograms (CFAEs) correlate with areas of slowed conduction and pivot points of reentrant wavelets. Ablation catheter 100, or a system of multiple ablation catheters which include ablation catheter 100, may be used to both identify the areas with AF wavelets reenter, as well as selectively ablate these areas causing wavelet reentry to stop and prevent the perpetuation of AF. Mapping may be performed by one or more ablation elements of ablation catheter 100, such as ablation elements comprising electrodes configured to deliver RF energy. In an alternative embodiment, one or more ablation elements of catheter 100 are further configured to deliver pacing energy, such as electrical energy configured to pace one or more portions of a human heart.

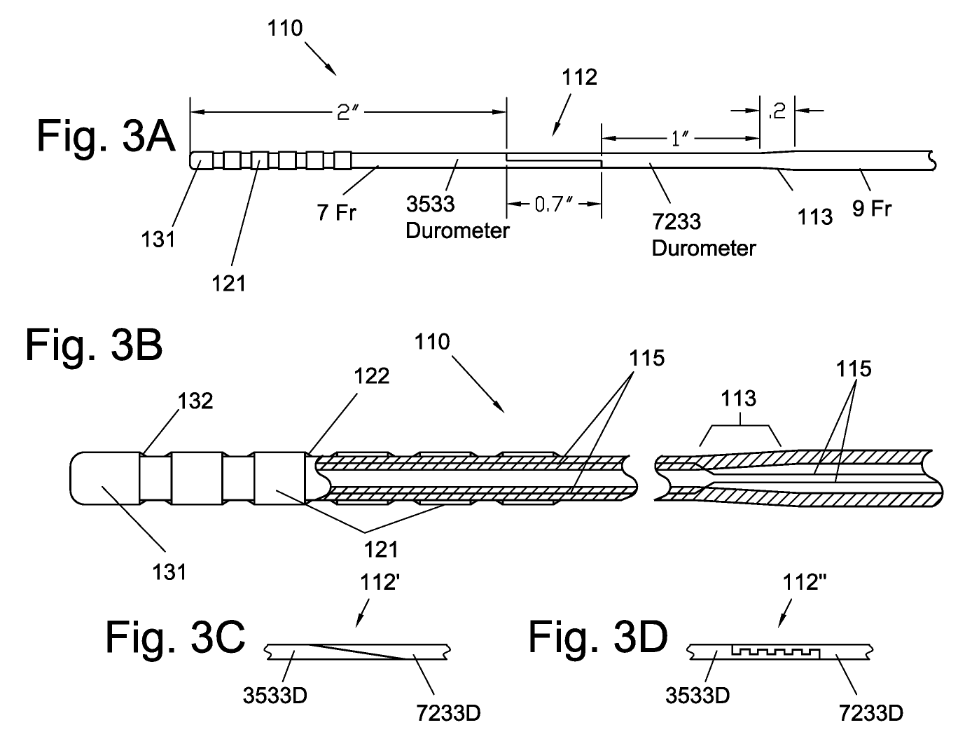

Referring now to FIGS. 3A and 3B, an ablation catheter of the present invention is illustrated. In FIG. 3A, a side-view of a distal portion of catheter shaft 110 is shown. Typical dimensions of various sections of catheter shaft 110 are also depicted. A proximal section has a larger diameter (e.g. 9 Fr) than the distal section (e.g. 7 Fr). In addition, the proximal section is stiffer (e.g. by using stiffer material such as 7233 durometer Pebax) that the distal section (e.g. Pebax of 3533 durometer). Shaft 110 is preferably made of one or more biocompatible materials commonly used in catheter construction, such that shaft 110 can be percutaneously introduced to the heart or other location within the body of a patient. Shaft 110 may be a laminate construction, such as a structure including: braiding such as stainless steel braid; embedded or attached members such as stiffeners and malleable (plastically deformable) members; liners such as Teflon liners which provide a low-friction surface for sliding members within shaft 110; and elongate tubes which reside within shaft 110.

As shown, the larger OD (9 Fr) portion of shaft 110 transitions to the smaller OD (7 Fr) portion at tapered joint 113. In a preferred manufacturing method, a 9 Fr tube, a 7 Fr tube, and a tapered tube which tapers from 9 Fr to 7 Fr, are bonded together, such as via heat bonding, adhesive bonding, or a combination of the two.

Also shown in FIG. 3A is a “staircase joint” 112, in which shaft 110 transitions from a stiffer material (e.g. 7233 durometer Pebax) to a more flexible material (e.g. 3533 durometer Pebax). Staircase joint 112 includes an overlap of the stiffer material with the more flexible material, such as with the two materials overlapping each other as shown in FIG. 3A. Staircase joint 112 may be constructed by cutting the step profile into two tubes (of different stiffness), and thermally bonding the two steps together. Alternatively or additionally, adhesive may be used. Staircase joint 112 provides a “hinge point” for deflection (steering), such as a deflection caused by advancement and/or retraction of a steering wire, not shown but described in detail in reference to subsequent figures herebelow. Staircase joint 112 may include an inserted elongate member, not shown but preferably an elastically biased member such as a Nitinol wire or stainless steel wire, or a malleable member. Steering of shaft 110 is typically 90° or more. Joint 112 avoids the need for creating a hinge point with a collar in the wall of and/or within a lumen of shaft 110. Joint 112 is configured such that deflection toward the stiffer material (i.e. towards the top of the page in FIG. 3A), is less (e.g. less curvature i.e. greater radius of curve) than the deflection toward the more flexible material (i.e. towards the bottom of the page in FIG. 3B). Numerous other geometries of joints which joint two dissimilar materials arranged to cause asymmetric deflection geometries may be incorporated, such as joint 112′ of FIG. 3C which includes a continuous taper between the two materials, and joint 112″ of FIG. 3D which includes a “toothed” joint construction.

Alternatively or additionally, shaft 110 may be modified with a stiffening member, not shown but located within the wall of or attached proximate an inner or outer wall of shaft 110, such as to create asymmetric deflection during steering and/or to provide a restoring force (e.g. a force configured to straighten or curve the distal portion of shaft 110). The stiffening member may be maintained proximate to shaft 110 with a braid or a liner. In a preferred embodiment, an elastic stiffener is attached to one side of shaft 110, such that deflection toward that side is less than deflection toward the opposite site. In another preferred embodiment, a plastically deformable stiffener is similarly attached, such that one or more curved shaped can be maintained until a restoring force is applied. Alternatively or additionally, shaft 110 may include an eccentric braid (absent or reduced in a portion of the full inner diameter of shaft 110), such that deflection toward the stiffer part of the braid is less than deflection toward the less stiff braid portion.

Referring back to FIG. 3A, at the distal end of shaft 110 is an ablation element, tip electrode 131, preferably made of platinum and having an atraumatic leading edge (such as to prevent perforation of the left atrium). Tip electrode 131 is adhesively bonded to shaft 110, and may further include a reduction of (including a portion of) its internal diameter such as via a crimp or swage on its proximal end to increase the attachment force to shaft 110. In alternative embodiment, tip electrode 131 and the distal end of shaft 110 have reverse, mating tapers (“Chinese finger grip”) such that an applied tension force causes increased attachment force. A crimp, swage or other geometry modification can also perform the function of removing a sharp edge on a tip (or shaft) electrode. Tip electrode 131 preferably has a length of 1 to 8 mm, and more preferably has a length of approximately 4 mm. Tip electrode 131 preferably has an inner diameter of 0.020″ to 0.300″ and more preferably has an inner diameter of approximately 0.094″. Tip electrode 131 typically has a surface area of approximately 33.7 mm², and preferably has a wall thickness of between 0.006″ and 0.010″, typically between 0.008″ and 0.010″. In an alternative embodiment, Tip electrode 131 has a wall thickness between 0.002″ and 0.020″.

Proximal to tip electrode 131 is a series of electrodes, shaft electrodes 121. In a preferred embodiment, 2 to 6 shaft electrodes are included. In an alternative embodiment, a single shaft electrode 121 is attached to shaft 110. Shaft electrodes 121 have an inner diameter configured to allow adhesive attachment of electrodes 121 to shaft 110 (e.g. closely matched diameters). In a preferred embodiment, one or both of the ends of electrodes 121 are swaged or crimped to increase the attachment force to shaft 110. The outer diameter of shaft electrodes 121 may be sized to be flush with the outer diameter of shaft 110, or in a preferred embodiment, the outer diameter of shaft electrodes 121 is slightly larger than the outer diameter of shaft 110 such that increased engagement with tissue can be achieved. In an alternative embodiment, shaft 110 includes a recessed portion on its outer diameter where shaft electrodes 121 are attached. Shaft electrodes 121 preferably have a length of 1 to 8 mm, and more preferably have a length of approximately 2 mm. Shaft electrodes 121 preferably have a diameter of 0.020″ to 0.300″ and more preferably have a diameter of approximately 0.094″ (e.g. when shaft 110 has a diameter of 0.090″). Shaft electrodes 121 typically have a surface area of approximately 29.5 mm², and preferably have a wall thickness of between 0.006″ and 0.010″, typically between 0.008″ and 0.010″. A first shaft electrodes 121 and a second shaft electrode 121 may have similar or dissimilar geometries and/or materials of construction. In a preferred embodiment, a first shaft electrode 121 and a second shaft electrode 121 are of different lengths or different thicknesses.

The shaft electrode 121 closest to tip electrode 131 is preferably located 1 to 8 mm from tip electrode 131, and more preferably 3 mm. The separation between shaft electrodes 121 is preferably 1 to 8 mm, and more preferably 3 mm. Each of the ablation elements mounted on shaft 110, is preferably a platinum electrode configured to deliver unipolar energy or bipolar energy (e.g. bipolar energy between adjacent electrodes or any pair of electrodes. Alternatively or additionally, one or more ablation elements may be an electrode constructed of platinum-iridium, gold, or other conductive material. Alternatively or additionally, the ablation elements may deliver another form of energy, including but not limited to: sound energy such as acoustic energy and ultrasound energy; electromagnetic energy such as electrical, magnetic, microwave and radiofrequency energies; thermal energy such as heat and cryogenic energies; chemical energy; light energy such as infrared and visible light energies; mechanical energy; radiation; and combinations thereof.

Shaft electrodes 121 and tip electrode 131 preferably include at least one temperature sensor such as a thermocouple. In a preferred embodiment, each electrode includes at least two thermocouples, such as two thermocouples mounted (e.g. welded) to the ID of each electrode, separated by 180°. In an alternative embodiment, three or more thermocouples are mounted to the ID of one or more electrodes, the thermocouples mounted at locations equidistant from each other. In another alternative embodiment, two or more thermocouples are mounted in an eccentric geometry, such as a geometry relating to one or more particular deflection geometries of the shaft, such as a first thermocouple located on the outside of the curve of a first deflection geometry, and a second thermocouple located on the outside of the curve of a second deflection geometry. In another alternative embodiment, one or more thermocouples are potted into an electrode wall such that the thermocouple is in direct contact with tissue during ablation. Signal wires, not shown, attach to the electrodes as well as the thermocouples, for delivering energy to the electrodes as well as transmitting information signals (e.g. temperature levels) back to the handle of the ablation catheter to which shaft 110 is attached.

Referring now to FIG. 3B, a partial cross-section of catheter shaft 110 of FIG. 3A is shown. In order to generate the asymmetric deflection described in reference to FIG. 3A (noting that staircase joint 112 is not shown), two steering wires 115 are included within the OD of shaft 110. In the larger diameter portion (e.g. 9 Fr portion), steering wires 115 “free float” within a lumen of shaft 110. At a point proximate (e.g. distal to) tapered joint 113, the steering wires 115 are fixedly attached to or embedded within shaft 110 (e.g. between a braid and shaft 110 and/or between a liner and shaft 110, braid and liner not shown but described in detail in description of subsequent figures herebelow). This configuration of the steering wires 115 results in one or more improvements including but not limited to: creation of a strain relief such as when shaft 110 is in tension (versus securing with a anchoring band which may create an undesired failure point during tensile loading); an increase in torque response of the distal portion of shaft 110; reduced “whipping” (undesired rotations or other undesired movement of a distal portion of a catheter while the proximal end of the catheter is applied with a torsional force); reduced “snaking” (deflection of an undesired, long portion of a catheter shaft, including deflection of the entire shaft); and combinations of these.

Also shown in FIG. 3B is tip electrode 131 and shaft electrodes 121. In addition to adhesive applied to the ID of each electrode, and crimping, swaging or otherwise modifying of one or more ends, each of which has been described above in reference to FIG. 3A, a fillet material, fillet 132 for tip electrode 131 and fillet 122 for shaft electrodes 121 may be included. Fillet material is preferably an adhesive, configured to further secure each electrode as well as eliminate a sharp edge at each electrode end. Alternatively, the fillet material may be a polymer, such as the Pebax shaft material, the fillet formed by adding Pebax and/or reflowing Pebax material with heat.

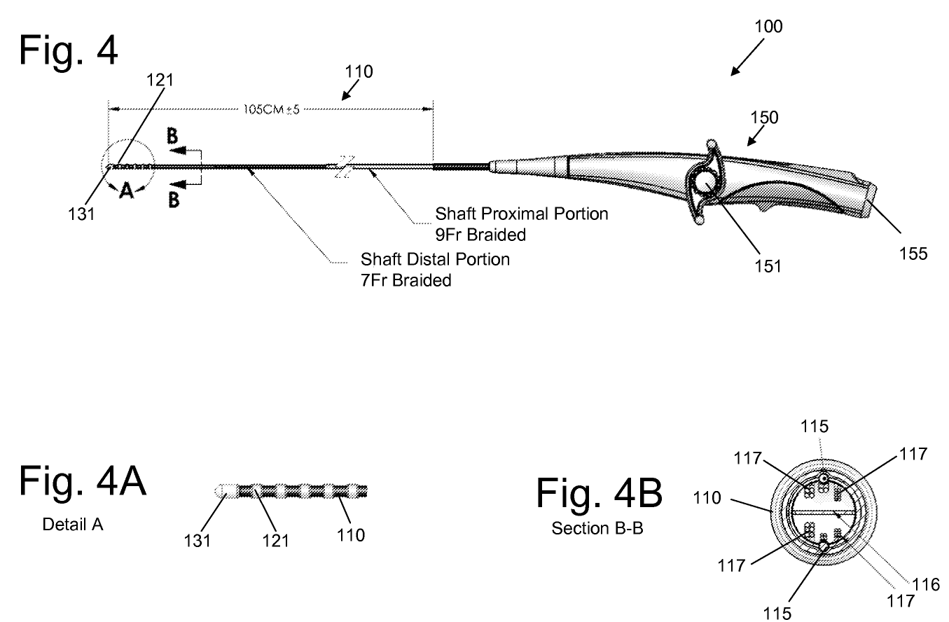

Referring now to FIGS. 4, 4A, 4B, 4C, 4D and 4E, an ablation catheter of the present invention is illustrated. In FIG. 4, a side-view of ablation catheter 100 is shown. Typical dimensions of various sections of ablation catheter 100 are also depicted. Shaft 110 includes a 9 Fr proximal portion and a 7 Fr distal portion, both of which are preferably braided. Alternative shaft reductions may be employed, such as an 8 Fr to 6 Fr transition, or other transitions preferably including a reduction of approximately 2 Fr. Braiding comprises typically a stainless steel flat wire and/or a nylon strand braiding material, although a wide variety of materials and cross-sectional geometries can be used for braiding. The stainless steel flat wire is typically 0.001″×0.003″ type 304 stainless steel, or equivalent. Braiding parameters preferably range from 40 ppi to 80 ppi. In another preferred embodiment, braiding of 80 ppi in the proximal portion of shaft 110 transitions to 60 ppi and then 40 ppi in the distal portion, such as to create a relatively constant torque transition during rotation.

Ablation catheter 100 includes handle 150 which includes an electrical connector, jack 155, which is electrically connected via multiple signal wires (not shown) to shaft electrodes 121 and tip electrode 131. Handle 150 further includes knob 151, which is operably attached to one or more steering wires, also not shown but described in detail throughout this application. Rotation of knob 151 causes deflection of the distal portion of shaft 110, such as deflections in one to four directions, with symmetric and/or asymmetric deflection geometries. Alternative or additional knobs may be included, such as a knob attached to a control wire which is further attached to a stiffening member, such as a stiffening member used to change the curve of a distal portion of shaft 110.

In FIG. 4A, a side view of the distal end of shaft 110 is illustrated (detail A of FIG. 4), including shaft electrodes 121 and tip electrodes 131. While the separations between each electrode are shown as relatively similar, dissimilar separation distances may be employed. While the lengths of shaft electrodes 121 are shown as relatively similar, dissimilar electrode lengths may be employed.

In FIG. 4B, a cross sectional view of shaft 110 is illustrated (section B-B of FIG. 4). Included within shaft 110 is guide plate 116, an elongate plate constructed of an elastic material such as stainless steel or Nitinol. Steering wires 115 are also shown, located 180° from each other and fixedly attached or embedded to shaft 110. The axis formed between the centers of each steering wire 115 is perpendicular to the longer axis of guide plate 116. In this construction, deflections in the plane of guide plate 116 are resisted (i.e. guide plate 116 has a preferred bending direction due to the high aspect ratio of its width versus height). Guide plate 116 further improves lateral stiffness of shaft 110. Guide plate 116 is fixedly attached (e.g. adhesive attachment) within shaft 110 near its distal end (within or near tip electrode 131) and travels proximally 1″ to 8″, preferably 5″ and also preferably to a location more proximal than the transition between the 7 Fr shaft and the 9 Fr shaft. Guide plate 116 is preferably not attached to any steering wire or steering mechanism.

Also shown in FIG. 4B are multiple signal wires 117, shown grouped in multiple bundles, which transmit energy, such as RF energy, to the ablation elements of catheter 100 such as tip electrode 131 and shaft electrodes 121. Signal wires 117 also receive signals from one or more sensors, such as pairs of thermocouples mounted to each electrode. Signal wire sizes and function are described in detail throughout this application, and specifically in reference to FIG. 1. In a preferred embodiment, tip electrode 131 is attached to two 36 gauge wires and shaft electrodes 121 are each attached to a 36 gauge wire and a 40 gauge wire. Tip electrode 131 is preferably configured to deliver up to 45 Watts of RF power, utilizing the two 36 gauge wires. Shaft electrode 121 is configured to deliver up to 20 watts of RF power, utilizing the one 36 gauge wire.

In FIG. 4C, a side view of a preferred sub-assembly of shaft 110 is illustrated, with typical dimensions shown. The distal end (the 1.0″ segment) is trimmed in manufacturing, and the tip electrode is attached. The subassembly of shaft 110 includes shaft proximal portion 110a, preferably Pebax at 5533 to 7533 durometer (typically 7533 durometer); and shaft distal portion 110b, preferably Pebax at 3533 to 4533 durometer (typically 3533 durometer), or at least of a material more elastic that the material of shaft proximal portion 110a. Shaft proximal portion 110a is fixedly attached (e.g. via thermal bond) to shaft distal portion 110b at staircase joint 112. The subassembly of shaft 110 includes braid 118, as has been described hereabove.

In FIG. 4D, side sectional view of detail C of FIG. 4C is illustrated. The distal end of shaft distal portion 110b is constructed of stiffer material than the more proximal portion (e.g. 5533 durometer versus 3533 durometer). The majority of the 5533 portion is trimmed in manufacturing, however a small amount remains which is later fixedly attached to tip electrode 131. The increased durometer provides a more stable platform for an adhesive bond, as well as for a mechanical engagement such as a crimp or swage. During manufacturing, a metal ring, anchor ring 144 is placed at the junction of the 5533 durometer shaft and the 3533 durometer shaft as shown. Shaft distal portion 110b includes liner 118, such as a Teflon liner, which is placed such that one or more steering wires, not shown, are sandwiched between liner 118 and shaft 110b. In a preferred embodiment, liner 118 travels proximally into shaft proximal portion 110a. Anchor ring 118 applies additional retaining force to prevent steering wire movement. Anchor ring 118 spans from the Pebax 3533d material to the Pebax 5533d material.

In FIG. 4E, an end cross sectional view of section D-D of FIG. 4C is illustrated. Section D-D is positioned within staircase joint 112, and indicates a preferred construction where the stiffer shaft proximal portion 110a occupies 150° to 170° of the diameter of the shaft, and the more flexible shaft proximal portion 110b occupies 190° to 210° of the diameter. In an alternative embodiment, shaft proximal portion 110a occupies 150° to 180° of the diameter of the shaft. The eccentric mating of materials in staircase joint 112 produces asymmetric, stable deflection geometries. FIG. 4E depicts a laminate construction including liner 143, braid 118 and shaft proximal portion 110a and shaft distal portion 110b. Positioned between liner 143 and shaft proximal portion 110a is a first steering wire 115a, and positioned between liner 143 and shaft distal portion 110b is a second steering wire 115b. Also included are signal wires, connecting the ablation elements to a jack on a handle mounted to the proximal end of shaft 110, signal wires, ablation elements, handle and jack not shown but described in detail throughout this application.

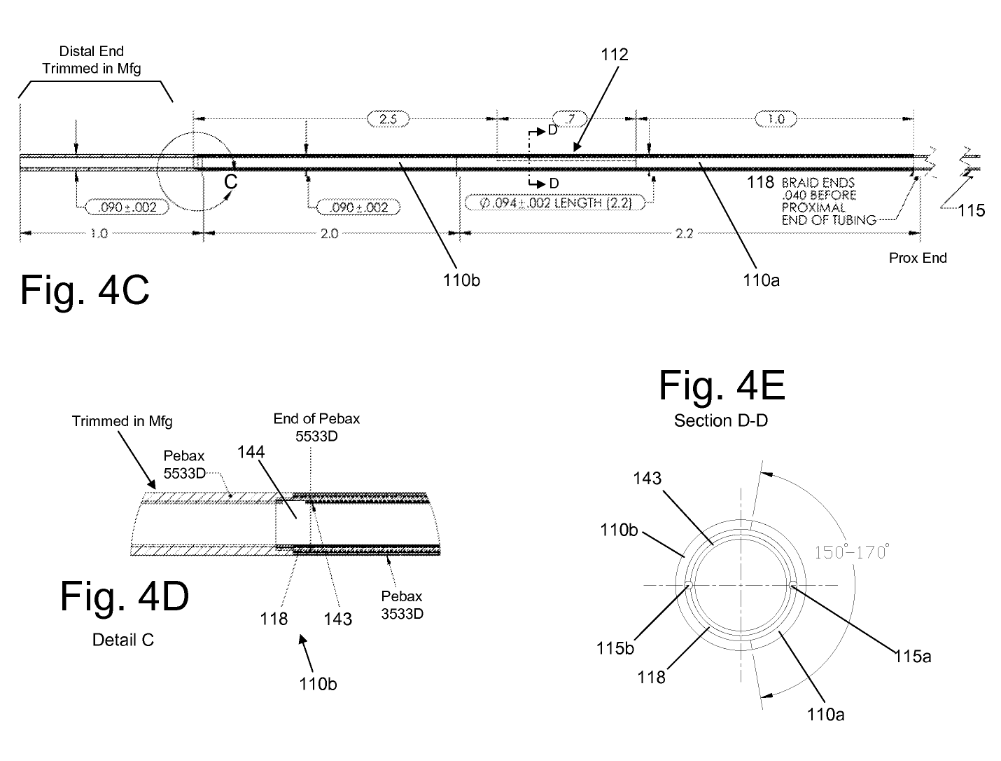

Referring now to FIG. 5, a schematic view of an ablation system of the present invention is illustrated, and an enlarged view of the distal end of the ablation catheter of FIG. 5 is shown in FIG. 5A. Ablation system 10 includes ablation catheter 100′ and fluid delivery device 500. Ablation catheter 100′ is fluidly attached to fluid delivery device 500 such that cooling fluid can flow past the internal and/or external walls of one or more of the electrodes of ablation catheter 100′ as well as to locations proximate tissue to be, being or having been ablated. Not shown, but preferably included in ablation system 10 is an RF generator which electromechanically attaches to ablation catheter 100′ to provide power to distal tip electrode 431 and band electrodes 121, as well as receives signals from temperature sensors, not shown but preferably thermocouples integrally mounted to distal tip electrode 431 and band electrodes 121. Also not shown but preferably included in ablation system 10 are an ECG monitoring unit connected to ablation catheter 100′ via an isolation device as shown and described in reference to FIG. 1A hereabove; a visualization device such as a fluoroscopy unit and/or an ultrasound monitor and probe; and other equipment common to an electrophysiology lab, catheterization lab, operating room or other patient treatment area of a hospital or other healthcare facility configured to ablate tissue such as cardiac tissue, tumor tissue, or other tissue to be denatured or removed in a patient treatment procedure.

Distal tip electrode 431, shown in FIG. 5A in a side sectional view of an enlarged portion of the distal end of shaft 110 of ablation catheter 100′, is preferably made of platinum, as are band electrodes 121, and are attached to shaft 110 in a similar manner as described hereabove. Distal tip electrode 431 includes walls 437 which surround chamber 410, and defines at its distal end, exit hole 431. A fluid delivery tube 405 enters the chamber 410 and terminates in the proximal section of chamber 410. Fluid delivery tube 405 travels proximally through shaft 110 and handle 150 providing a fluid connection to tubing 451 and luer 450 such that a fluid delivery device may deliver fluid into chamber 410 and exiting exit hole 431. A fluid barrier, plug 404, seals around fluid delivery tube 405 preventing fluid from exiting chamber 410 into the lumen of shaft 110. Plug 404 made be made from one or more sealing materials such as a metal, plastic or elastomer, and preferably an epoxy.

Fluid delivery system 500 is fluidly attached to luer 450 such that cooling fluid can flow through a lumen of shaft 110, into chamber 410 providing heat exchange (cooling) to the internal side of wall 410, and out of exit port 431 to locations neighboring the outside of wall 410 as well as tissue to be, being, or having been ablated. In a preferred embodiment, fluid delivery begins prior to delivery of ablation energy by distal tip 431, such as approximately 3 seconds before initiation of energy delivery. In another preferred embodiment, fluid delivery begins after delivery of ablation energy by distal tip 431, such as approximately 5 seconds after cessation of energy delivery.

The fluid delivery system 500 of FIG. 5 consists of collapsible bag 501 preferably filled with a biocompatible fluid such as saline solution. Bag 501 is surrounded by pressurization cuff 502, configured to apply pressure to bag 501 as a driving force to infuse fluid into catheter 100′ via tubing 503. Bag 501 and pressurization cuff are shown attached to pole assembly 505, such as an IV pole used in healthcare facilities. Fluid delivery system 500 is configured to deliver a flow rate in a range to maximize the heat exchange benefit to one or more electrodes of ablation catheter 100′. Depending on the geometry of the fluid flow pathway, including the contacting portions to the electrodes such as the geometry of chamber 410, the flow rate is selected to maximize a continuous flow of fluid near the boundaries between the wall 437 and the cooling fluid. Heat exchange is increased by increasing the difference in temperature of the electrode surface (or tissue surface) and the temperature of the cooling fluid (ΔT). Flow rates which create stagnant flow zones are avoided, such as a flow rate which causes a vortex that results in the majority of the flow to occur in the center of chamber 410 (i.e. causing slow or minimal flow at the boundary with wall 437). Flow rates can be chosen that cause turbulent flow to be generated, such turbulent flow causing constant mixing of the fluid in these boundary areas with cooler fluid (increasing ΔT).

Alternative forms of fluid delivery system 500 may be used in substitution of or in addition to fluid delivery system 500 of FIG. 5. Examples of fluid delivery systems include but are not limited to: a constant pressure pump such as a pump including a pressurization chamber and a pressure regulated output; a constant flow pump such as a syringe driver pump; a peristaltic pump; a gravity feed drip controller; and the like. The fluid delivered by fluid delivery device 500 is preferably saline, and is maintained at a temperature lower than ablation temperature, preferably lower than body temperature, and more preferably lower than room temperature, such as a fluid that has been refrigerated. In the configuration of ablation catheter 100′ of FIGS. 5 and 5A, the cooling fluid exits ablation catheter 100′ and enters the body of the patient, such that a biocompatible fluid must be used. In alternative configurations of an ablation catheter of the present invention, the cooling fluid is completely maintained within one or more lumens or electrode chambers of the ablation catheter (i.e. circulated through a catheter with no exit holes), such that a biocompatible fluid is not necessary. In a preferred embodiment, the cooling fluid is maintained at room temperature. In another preferred embodiment, the cooling fluid is maintained at a temperature less than room temperature, such as a saline bag which has been obtained from a refrigerator or wherein fluid delivery system 500 includes a cooling device in relative contact with the cooling fluid during the procedure.

Shaft 110 is preferably of similar construction to shaft 110 of FIGS. 4A through 4C. Shaft 110 includes one or more lumens for delivery of the cooling fluid received from fluid delivery system 500 to locations proximate one or more of its electrodes. These or other lumens may also be included to perform one of more functions including but not limited to: over a guidewire delivery or manipulation; transport of electrical power and sensor wires to tip electrode 431, shaft electrode 121 and/or another transducer or sensor of the ablation catheter; support and longitudinal translation of one or more control wires or shafts such as a deflection pull wire or an electrode array advancable and/or retractable control shaft; or for other purposes. The electrodes of ablation catheter 100′ preferably include one or more temperature sensors, not shown but typically one or more thermocouples integrally mounted to each electrode and connected to one or more wires that travel proximally to handle 150 for attachment to an energy delivery system. In a preferred embodiment, tip electrode 431 includes two, three or four thermocouples, located circumferentially at 180°, 120° or 90° spacing, respectively. Temperature monitoring circuitry may monitor two or more temperature sensors, and adjust power delivery based on one sensor (e.g. the highest reading) and/or multiple sensors. Also not shown are power delivery wires, attached to each electrode and traveling proximally to handle 150 for attachment to an energy delivery system, preferably for delivery of monopolar and bipolar RF energy.

The ablation catheters and cooling devices of the present invention, as have been described in reference to FIGS. 27 and 27A and numerous other figures of this application, are configured to safely and effectively deliver more power than ablation catheters without a cooling system. In the ablation catheter of FIG. 5 and subsequent figures, RF energy may be safely delivered at wattages over 30 Watts, preferably up to 45 Watts and greater, and potentially up to approximately 100 Watts, such as by avoiding overheating of tissue or blood during energy delivery by effective cooling of the electrodes and or neighboring tissue and blood.

In an alternative embodiment, fluid delivery system includes an electronic valve, and a component of the system, such as the RF generator, opens the valve during delivery of ablation energy. In another preferred embodiment, the valve is opened prior to delivery of ablation energy, such as 3 seconds prior, and the valve is maintained open after delivery of ablation energy, such as 5 seconds after. In an alternative embodiment, the cooling fluid is administered during particular (i.e. not all) ablations, such as when a temperature threshold has been exceeded, an unknown state is entered (e.g. due to the loss of signal from a thermocouple), a warning or alert condition is encountered, a particular power or other energy delivery setting is selected, or by the occurrence of another event or condition common to a tissue ablation procedure.

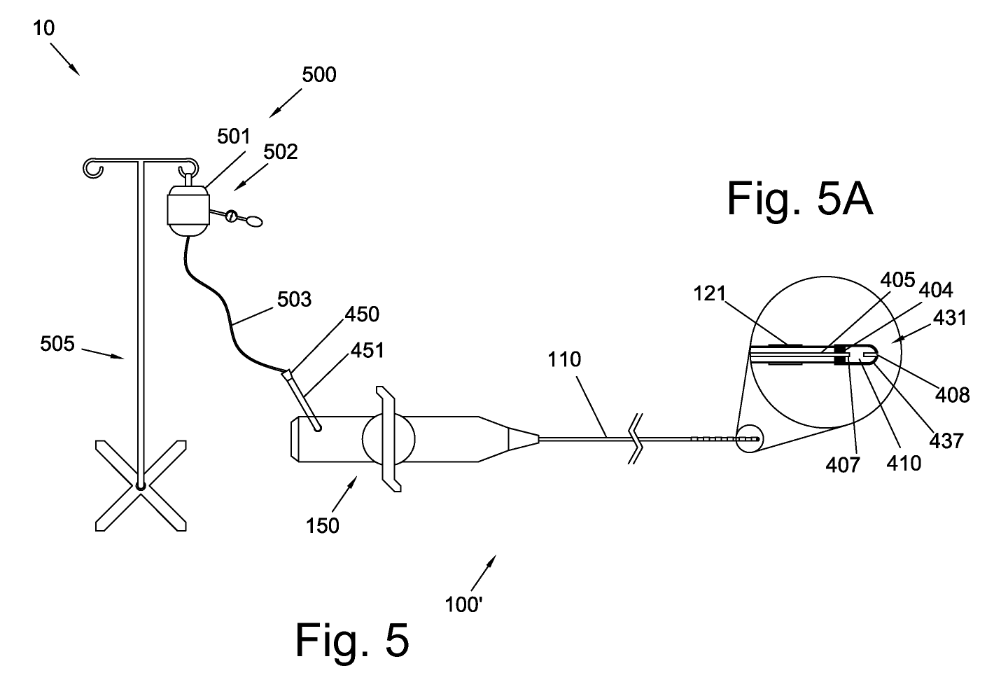

Referring now to FIGS. 28A, 28B and 28C, multiple views of a preferred embodiment of a tip electrode of the present invention are illustrated. FIG. 6A is a perspective view of tip electrode 431a which includes slit 408. FIG. 6B is an end sectional view of electrode 431A depicting slit 408 and wall 437. FIG. 6C is a side sectional view (Section A-A of FIG. 6B) indicating preferred dimensions of tip electrode 431a. Tip electrode 431a is preferably made of platinum and its ID (approx 0.094 inches) is sized for mechanical fixation to a catheter shaft, as has been described in detail hereabove. Chamber 410 and slit 408 geometries are configured such that a flow rate of approximately 0.1 through 8.0 ml/min, preferably 0.9-8.0 ml/min provides sufficient heat transfer (cooling) to safely and effectively deliver high power to heart or other body tissue, such as in the treatment of atrial fibrillation. In the flow device configuration of FIG. 5, a pressure approximating 500 mmHg can be used to achieve the desired flow rate. Slit 408 is sized to slow down or otherwise limit flow through chamber 410. Typical dimensions of slit 408 are 0.004-0.006″ wide and 0.026+/−0.002″ deep. The depth of slit 408 is chosen such that when the distal end of tip electrode 431 is placed into tissue, a portion of slit 408 is not occluded by that tissue, allowing cooling fluid to pass through slit 408, typically into the circulating blood of a chamber of the patient's heart such as the left atrium. Wall 437 has a preferred thickness of 0.008-0.010″ which has been shown to effectively transfer heat by rapidly reaching ablation temperature during energy delivery and rapidly cooling during a non-energy delivery time portion. Tip electrode 431a has a length of approximately 0.20-0.26″, typically 0.236″ as shown.

Multiple different geometries of an exit port such as slit 408 may be integrated into tip electrode 431a, as are described by example only in reference to subsequent figures. Additional cooling fluid exit holes may also be included, such as one or more holes that exit the side wall of tip electrode 431a. These exit holes are configured to maximize flow at the boundary between the cooling fluid and the electrode wall. Additional system parameters can also be modified to improve heat transfer such as fluid flow rate and fluid viscosity. Ablation systems that include electrodes with the surface area, mass and other geometric properties similar (or larger) to those depicted in FIGS. 28A-C can achieve increased safety and effectiveness with the inclusion of the electrode exit ports such as slit 408, electrode housing geometry such as chamber 410, and fluid delivery system 500 of FIG. 5, all as are described throughout this application.

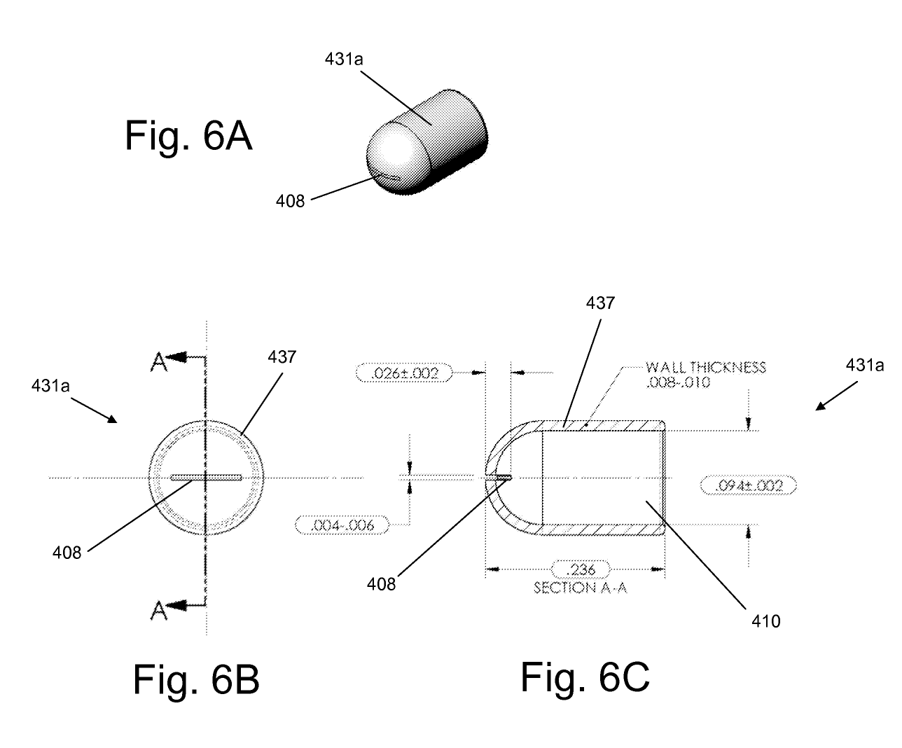

Referring now to FIGS. 7A and 7B, an ablation catheter and two preferred ablation methods of the present invention are illustrated. Ablation catheter 100′ includes tip electrode 431a, of similar construction to tip electrode 431a of FIG. 6A-C. Tip electrode 431a is attached to shaft 110, which further includes shaft electrodes 121. Typically, the methods of FIGS. 7A and B are performed in using a percutaneous catheter procedure in a chamber of the heart, such as the right atrium, and the distal portion of catheter 100′ is surrounded by circulating blood. In other ablation locations within the body, in percutaneous, laparoscopic or open surgical procedures, the catheter may be surrounded by another body fluid, body tissue, or a gas such as carbon dioxide or air (e.g. in laparoscopic procedures).

Referring specifically to FIG. 7A, a preferred method is shown wherein the distal portion of ablation catheter 100′ is positioned relatively orthogonal to the patient's tissue to be ablated, with the distal end tip electrode 431a in contact with the tissue. The ends of slit 408 are shown above the tissue boundary, and saline is shown exiting slit 408 and “pooling” at the ablation site. The condition depicted in FIG. 7A is typically present just prior to, during, or soon after ablation energy delivery, such as to allow greater RF energy to be delivered, without causing charring, creating a blood embolus, or other undesired clinical event. Tip electrode 431a may be delivering monopolar energy, such as through a ground pad as has been described hereabove, and/or delivering bipolar energy such as in combination with one or more shaft electrodes 121.

Referring specifically to FIG. 7B, a preferred method is shown wherein the distal portion of ablation catheter 100′ is positioned relatively parallel to the patient's tissue to be ablated, with the distal end tip electrode 431a and multiple shaft electrodes 121 in contact with the tissue. Slit 408 is shown above the tissue boundary, and saline is shown exiting slit 408 and “pooling” at the location neighboring tip electrode 431a. The condition depicted in FIG. 7A is typically just prior to, during, or soon after ablation energy delivery, such as to allow greater RF energy to be delivered, without causing charring, creating a blood embolus, or other undesired clinical event. Tip electrode 431a may be delivering monopolar energy, such as through a ground pad as has been described hereabove, and/or delivering bipolar energy such as in combination with one or more shaft electrodes 121.

It should be appreciated that other orientations of the distal end of ablation catheter 100′ can be used, such as when a most distal portion is parallel and in contact with tissue, and a more proximal distal portion is at an angle with the tissue, such as an angle of approximately 22°, 45°, 67° or 90°, such as a configuration where more distal shaft electrodes are in contact with tissue and more proximal shaft electrode are not.

Referring now to FIGS. 30 A through D, multiple tip electrodes with exit ports of the present invention are illustrated. Each of these tip electrodes have a proximal end configured for attachment to a catheter shaft as has been described in detail hereabove, and are preferably constructed, at least in their energy delivery portions, of platinum. When attached to the catheter shaft, the tip electrodes are configured to be in fluid communication with fluid delivery means provided by the catheter shaft.

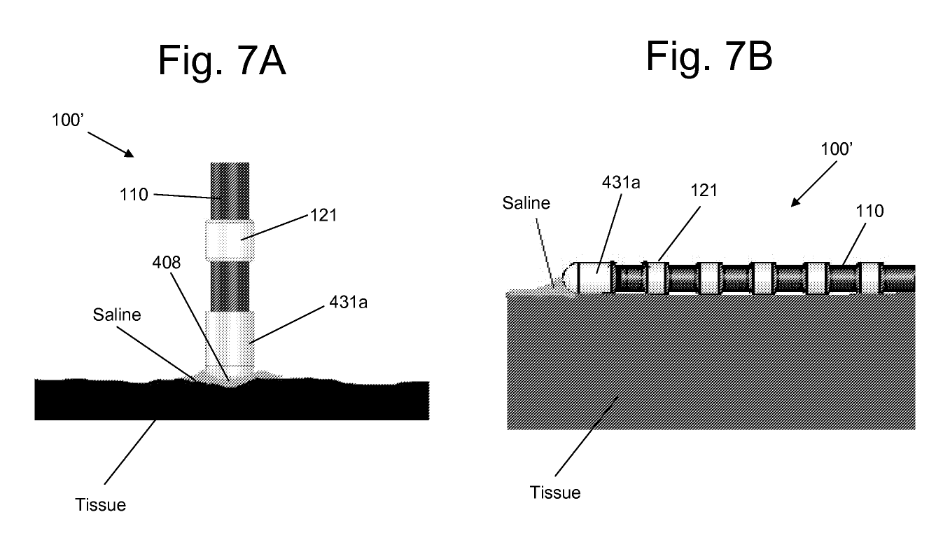

Referring specifically to FIG. 8A, a perspective view of tip electrode 431b is shown. Tip electrode 431b includes exit hole 401 and recesses 402a and 402b, positioned relatively perpendicular to one another and configured to allow fluid to pass through recess 402a and/or 402b when the distal end of tip electrode 431b is pressed into tissue. Additional or alternative recess configurations may be incorporated. Referring now to FIG. 8B, a perspective view of tip electrode 431c is shown. Tip electrode 431c includes exit hole 401. At the distal end of exit hole 401 is chamfer 403, sized and configured to reduce the likelihood of exit hole 401 occluding when the distal end of tip electrode 431 is pressed into tissue, such as by reducing the sealing surface area as tip electrode 431 is pressed into tissue.

Referring specifically to FIGS. 30C and 30D, side and end views, respectively, of tip electrode 431d are shown. The proximal end of tip electrode 431e includes an opening to channel 431, the opening configured to be attached to a source of cooling fluid from an ablation catheter shaft. Channel 431 is configured in a corkscrew or spiral geometry wherein the flow path preferably travels at a diameter in proximity to the exterior surface of tip electrode 431d. The distal end of channel 431 is exit hole 401, configured to allow cooling fluid to controllable exit tip electrode 431d. Exit hole 401 may be configured in the circular geometry shown, or multiple other configurations such as the slit design described in reference to tip electrode 431a of FIG. 6A-C. Channel 433 has a diameter and profile configured to prevent slow flow at its outer edges. Channel 433, and the other surfaces of any of the electrodes of the present invention such as internal and external surfaces of electrode walls, may include one or more of: liners (including partial liners) such as teflon liners; coatings such as hydrophilic or hydrophobic coatings; treatments such as surface energy modifications configured to improve boundary flow rates of the cooling fluid; texture modifications such as grooved or roughened surfaces configured to improve boundary flow rates and/or create turbulent flow, and other modifications configured to improve flow at a boundary layer, create turbulent flow, or otherwise increase heat exchange between the electrode and the cooling flow. The creation of turbulent flow can be beneficial due to the mixing of fluid at a boundary, with cooler fluid away from a boundary.

Referring specifically to FIGS. 30E and 30F, side and end views, respectively, of tip electrode 431e are shown. Within walls 437 of tip electrode 431e are multiple fins 432, traveling from at or near (as shown) the proximal end of tip electrode 431e to a location at or proximal to (as shown) exit hole 401. A thru-hole, not shown, may be included for over a guidewire delivery or manipulation. Fins 432 are configured to provide one or more of: an efficient heat sinking function as achieved with the combined surface area of the fins and exposed walls 437; high flow at all fin surfaces due to the small cross-sectional area of each defined channel. Fins 432 may be constructed of the same material as the electrode, such as platinum, or they may be constructed of a different material or combination of materials such as a material with a higher thermal conductivity such as copper or aluminum. When constructed of electrically conductive material, fins 432 may be attached to walls 437 creating an electrical connection, or an electrical isolation layer may be incorporated (to avoid passage of RF energy through fins 432). In all cases, a good thermal connection is desirable. Similar to the modifications described in reference to FIGS. 30C and D, the exposed surfaces of fins 432 and walls 437 may be modified to improve heat exchange (surface treatment, modification, etc) with the passing cooling fluid.

The tip electrodes of FIGS. 30A-F include various features that can be incorporated individually or in combination into the electrodes of the present invention. These tip electrodes may include additional fluid exit means, such as holes, slits or other openings at the distal end or side walls of the electrode. These fluid exit means are configured to improve flow conditions for improved heat exchange between the electrode and surrounding tissue and blood, and the cooling fluid of the present invention. Each of the tip electrodes may include one or more openings configured for over a guidewire delivery or manipulation.

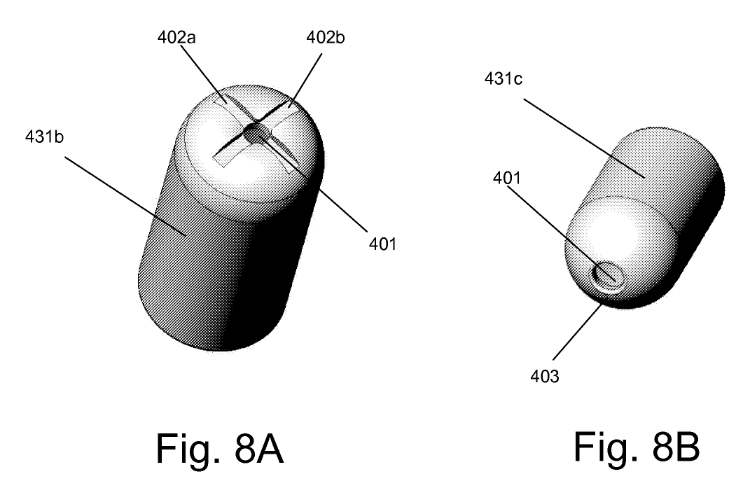

Referring now to FIGS. 31A through C, a side view, a sectional end view and a sectional side view, respectively, of a preferred embodiment of a shaft electrode of the present invention is illustrated. Shaft electrode 421 is configured for mounting on a shaft of an ablation catheter as has been described hereabove, and is preferably made of platinum. Shaft electrode 421a includes multiple side holes 412 exiting two circumferential portions of wall 436 as shown in FIG. 9B. Shaft electrode 421a includes an integrally mounted thermocouple 138, and may include additional thermocouples, not shown, but preferably of constantan and alloy 11 construction as shown in FIG. 9C. When mounted to an ablation catheter shaft, a number of holes 412, preferably a majority of the holes 412 are in fluid communication with fluid passing through the shaft, such that shaft electrode 421a transfers heat to the fluid.

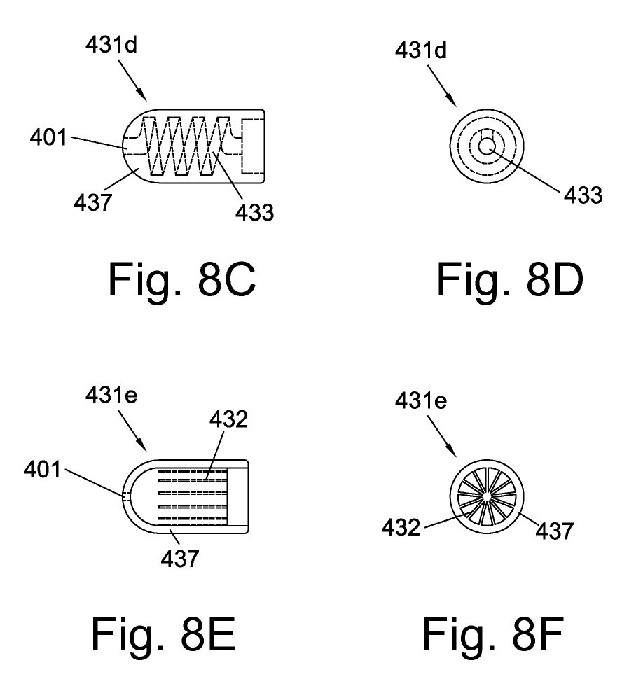

Referring now to FIG. 10, a side sectional view of a preferred embodiment of a shaft electrode of the present invention is illustrated. Shaft electrodes 421b′ and 421b″ are configured to be mounted to ablation catheter shaft 110 such that a portion of each reside within a fluid carrying lumen of shaft 110 and a portion resides external to shaft 110 such as to be placed in contact with tissue. With the construction shown, cooling fluid may pass through the inner and outer walls of a portion of electrodes 421b′ and 421b″ which results in increased transfer of heat. Electrodes 421b′ and 421b″ may deliver energy independently in monopolar or bipolar mode with another electrode (such as a return pad or other shaft electrode, or in combination by delivering bipolar energy between them.

Shaft 110 is preferably of similar construction to shaft 110 of FIGS. 4A through 4C. Shaft 110 includes one or more lumens for delivery of the cooling fluid received from fluid delivery system 500 to locations proximate one or more of its electrodes. These or other lumens may also be included to perform one of more functions including but not limited to: over a guidewire delivery or manipulation; transport of electrical power and sensor wires to a tip electrode, shaft electrodes 421b′ and 421b″ and/or another transducer or sensor of the ablation catheter; support and longitudinal translation of one or more control wires or shafts such as a deflection pull wire or an electrode array advancable and/or retractable control shaft; or for other purposes. The electrodes of the ablation catheter preferably include one or more temperature sensors, not shown but typically one or more thermocouples integrally mounted to each electrode and connected to one or more wires that travel proximally to a handle for attachment to an energy delivery system. In a particular embodiment, shaft electrodes 421b′ and 421b″ include two, three or four thermocouples, located circumferentially at 180°, 120° or 90° spacing, respectively. Also not shown are power delivery wires, attached to each electrode and traveling proximally to a handle for attachment to an energy delivery system, preferably for delivery of monopolar and bipolar RF energy.

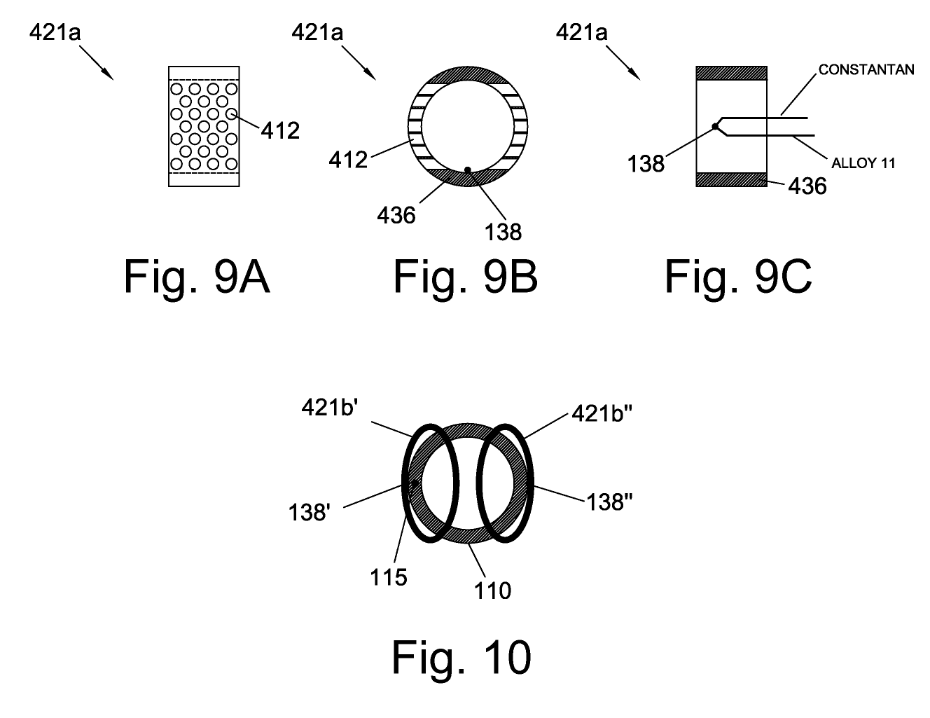

Referring now to FIG. 11, a perspective, not to scale view of an ablation catheter of the present invention is illustrated. Ablation catheter 100′ includes handle 150 which is fixedly attached to shaft 110 which includes tip electrode 431 and shaft electrodes 121, all preferably made of platinum and each including one or more thermocouples, not shown. Within a lumen of shaft 110 is fluid delivery tube 405 which is in fluid communication with tubing 451 and luer 450, configured for attachment to a cooling fluid delivery device as has been described in detail in reference to FIG. 5 hereabove. Tip electrode 431 includes walls 437 which define an internal space, chamber 410. Fluid delivery tube 405 passes through a sealing plug 404, into the distal end of chamber 410. Plug 404 made be made from one or more sealing materials such as a metal, plastic or elastomer, and preferably an epoxy. Also shown in FIG. 11 is a power delivery wire, wire 117, for transfer of monopolar and/or bipolar energy to tip electrode 431.

The portion of fluid delivery tube 405 residing within chamber 405 includes multiple side holes 406 which allow cooling fluid such as saline to be introduced into luer 450, travel through fluid delivery tube 405 into chamber 410 and exit through slit 408, preferably configured as described in reference to FIGS. 28A-C. Fluid exiting slit 408 is further utilized to cool the outer surface, especially the distal end, of tip electrode 431 as well as neighboring tissue. Side holes 406 are configured to create turbulent flow within chamber 410 such that stagnant or low flow areas are avoided along the internal side of walls 410. In combination with slit 408, side holes 406 further create sufficient back-pressure to create predictable flow rates based on driving pressure of the cooling delivery system, not shown.

Shaft 110 is preferably of similar construction to shaft 110 of FIGS. 4A through 4C. Shaft 110 includes one or more lumens for delivery of the cooling fluid received from fluid delivery system to locations proximate one or more of its electrodes. These or other lumens may also be included to perform one of more functions including but not limited to: over a guidewire delivery or manipulation; transport of electrical power and sensor wires to tip electrode 431, shaft electrode 121 and/or another transducer or sensor of the ablation catheter; support and longitudinal translation of one or more control wires or shafts such as a deflection pull wire or an electrode array advancable and/or retractable control shaft; or for other purposes. The electrodes of ablation catheter 100′ preferably include one or more temperature sensors, not shown but typically one or more thermocouples integrally mounted to each electrode and connected to one or more wires that travel proximally to handle 150 for attachment to an energy delivery system. In a preferred embodiment, tip electrode 431 includes two, three or four thermocouples, located circumferentially at 180°, 120° or 90° spacing, respectively. Also not shown are power delivery wires, attached to each electrode and traveling proximally to handle 150 for attachment to an energy delivery system, preferably for delivery of monopolar and bipolar RF energy.

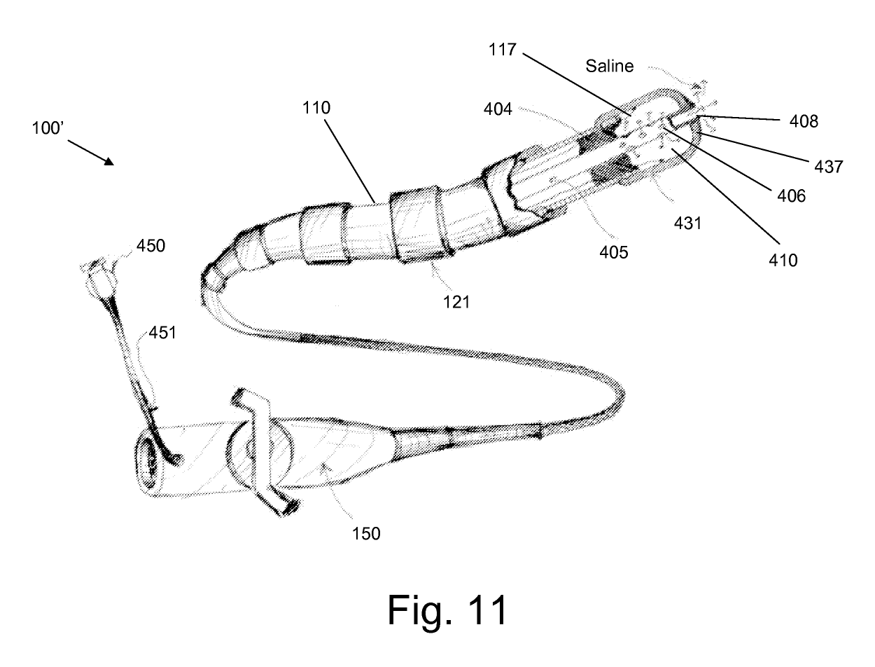

Referring now to FIG. 12, a perspective, partial cutaway view of the distal portion of an ablation catheter of the present invention is illustrated. Shaft 110 is preferably of the same construction and internal componentry as shaft 110 of FIG. 11. Shaft 110 includes shaft electrode 121 mounted to its outer diameter and tip electrode 431 mounted at its distal end, both preferably of platinum construction and both preferably including at least one temperature sensor such as a thermocouple. The distal end of the ablation catheter is of similar construction to that of the distal end of the ablation catheter of FIG. 11 with a different geometry flow tube 405. Flow tube 405 of FIG. 12 similarly terminates at a distal portion of chamber 410; however the distal portion of flow tube 405, within chamber 110, has a tapered diameter portion resulting in a smaller diameter tube at its distal end. In the larger diameter portion within chamber 110, side hole 406 is located and allows fluid to enter chamber 410 and absorb heat energy from walls 437 of tip electrode 431. Fluid exits chamber 410 via exit hole 401, shown as a circular hole. Fluid also exits flow tube 405, directly out of exit hole 401, without passing through chamber 410. The reduced diameter distal portion of flow delivery tube 405 helps limit flow through tube 405, as well as increase flow through side hole 406. While exit hole 401 is shown in FIG. 12 as a circular hole, other opening geometries can be used such as the slit geometry described in reference to FIGS. 6A-C.

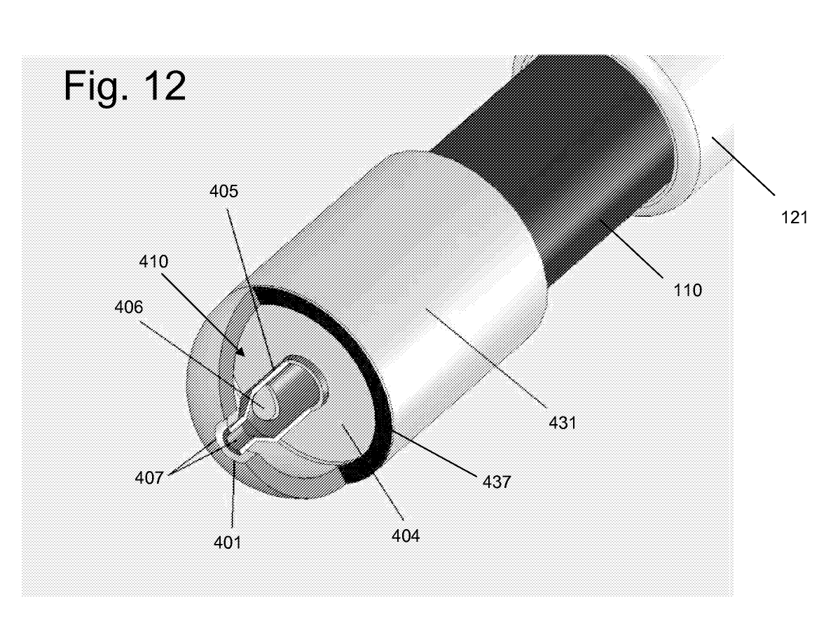

Referring now to FIGS. 13A and 13B, an end and side view, respectively, of the distal portion of an ablation catheter of the present invention is illustrated. Shaft 110 is preferably of the same construction and internal componentry as shaft 110 of FIG. 11. Shaft 110 includes multiple shaft electrodes 121 mounted to its outer diameter and tip electrode 431g mounted at its distal end, all electrodes preferably of platinum construction and all preferably including at least one temperature sensor such as a thermocouple. The distal end of tip electrode 431g includes an exit hole, slit 408a which is of similar width and depth to that of tip electrode 431a of FIGS. 6A-C, but has a curvilinear geometry as shown in FIGS. 13A and 13B. The curvilinear exit pathway creates a non-uniform flow of fluid exiting the chamber formed by walls 437 through slit 408a, as well as creating additional cross-sectional area of the exit portion and thus less flow resistance and cooling fluid contact area when compared to a design of a similar diameter electrode with a straight (non-curvilinear) slit.

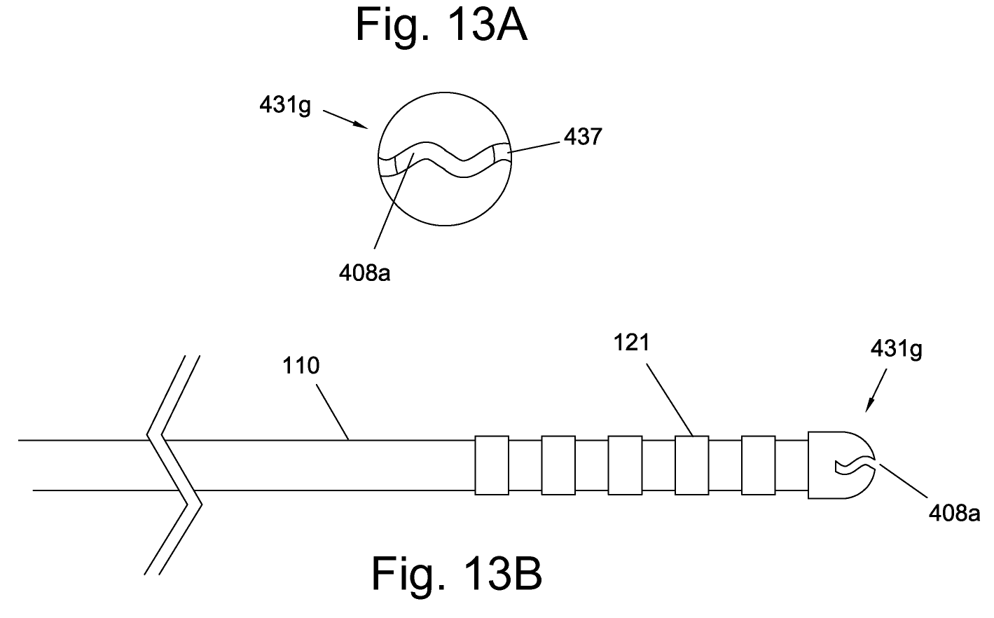

Referring now to FIG. 14, a side view of the distal portion of an ablation catheter of the present invention is illustrated. Shaft 110 is preferably of the same construction and internal componentry as shaft 110 of FIG. 11. Shaft 110 includes multiple shaft electrodes 421 mounted to its outer diameter and tip electrode 431 mounted at its distal end, all electrodes preferably of platinum construction and all preferably including at least one temperature sensor such as a thermocouple. The distal end of tip electrode 431 includes walls 437 which define an exit hole, slit 408 which is of similar geometry to that of tip electrode 431a of FIGS. 28A-C.

Shaft electrodes 421 include an exit hole 412 which is in fluid communication with fluid delivery tube 405 of shaft 110. Fluid delivery tube 405 has a distal end 407 that terminates at a location flush with, just proximal to, or just distal to (as shown) plug 404's distal end, such that fluid enters chamber 410 at its proximal portion. As cooling fluid is delivered to fluid delivery tube 405, as has been described in detail in reference to FIG. 5, cooling fluid passes through shaft electrodes 421 and tip electrode 431, removing heat from both shaft and tip electrodes.

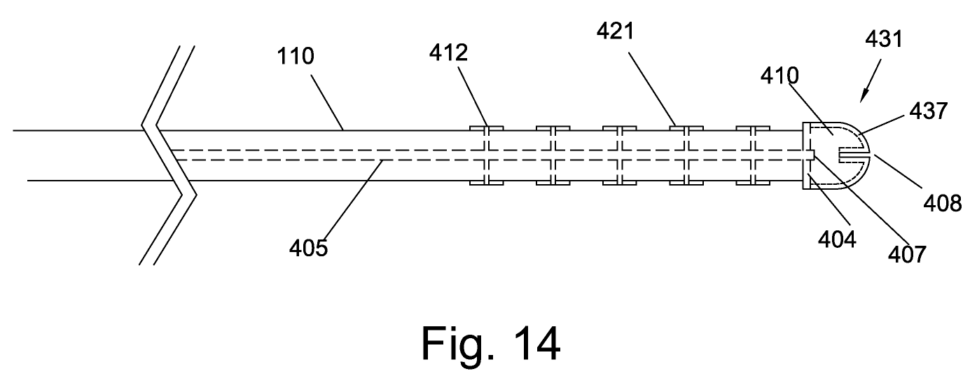

Referring now to FIG. 15, a perspective, not to scale view of an ablation catheter of the present invention is illustrated. Ablation catheter 100′ includes shaft 110, which is preferably of the same construction and internal componentry as shaft 110 of FIG. 11. Ablation catheter 100′ further includes handle 150 which is fixedly attached to shaft 110 which includes tip electrode 131 and shaft electrodes 121, all preferably made of platinum and including one or more thermocouples, not shown. Within a lumen of shaft 110 is first fluid delivery tube 405a which is in fluid communication with tubing 451 and luer 450, configured for attachment to a cooling fluid delivery device as has been described in detail in reference to FIG. 5 hereabove. Tip electrode 131 includes walls 137 which define an internal space, chamber 410. First fluid delivery tube 405 passes through a sealing plug 404, into the proximal end of chamber 410. Plug 404 may be made from one or more sealing materials such as a metal, plastic or elastomer, and preferably an epoxy.

Also in fluid communication chamber 410 is second fluid delivery tube 405b, also passing through sealing plug 404 into chamber 410. The opposite end of second fluid delivery tube 405b passes through a wall of shaft 110. Cooling fluid such as saline is introduced into luer 450, travels through first fluid delivery tube 405a into chamber 410, and exits through second delivery tube 405b to a location outside of the ablation catheter 110′. As the cooling fluid passes through chamber 410, heat is absorbed from walls 410 of tip electrode 131. Fluid exiting second delivery tube 405b cools neighboring tissue, blood, and one or more shaft electrodes 121, such as the most proximate shaft electrode 121.

Referring now to FIGS. 38A and B, side sectional views of the distal portion of an ablation catheter of the present invention is illustrated. Shaft 110 is preferably of the same construction and internal componentry as shaft 110 of FIG. 11. Shaft 110 includes multiple shaft electrodes 121 mounted to its outer diameter and tip electrode 431 mounted at its distal end, all electrodes preferably of platinum construction and all preferably including at least one temperature sensor such as a thermocouple. The distal end of tip electrode 431 includes walls 437 which define an exit hole 401.

Shaft 110 surrounds fluid delivery tube 405, which travels proximally to a fluid connection port, not shown but located on the proximal end of the ablation catheter and configured for attachment to a cooling fluid delivery system. Tip electrode 431 includes wall 437 which defines proximal chamber section 431a and distal chamber section 431b. Fluid delivery tube 405 has a distal end 407 that terminates at a location flush with, just proximal to, or just distal to (as shown) the distal end of sealing plug 404, such that fluid enters chamber proximal portion 410a. Located at a point between chamber proximal portion 410a and chamber distal portion 410b is a temperature controlled valve assembly 440.

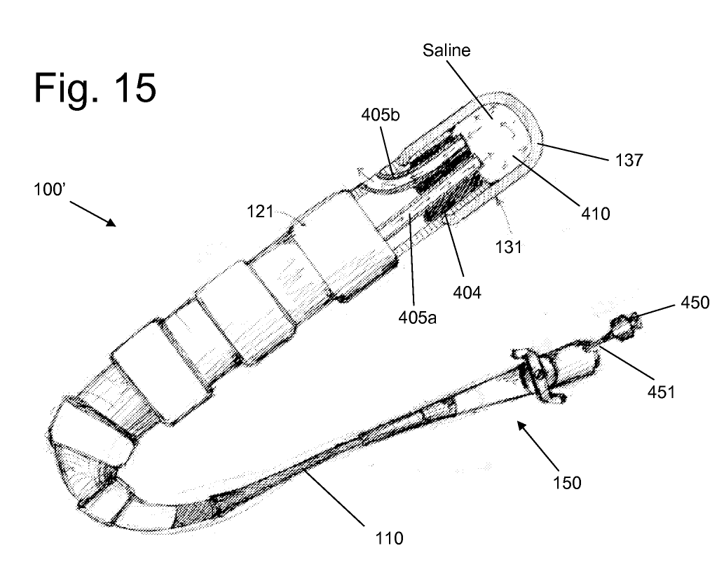

Valve assembly 440 is mechanically fixed to tip electrode 431 with support members 444, preferably rigid struts mechanically fixed and one end to valve assembly 440 and at the other end to wall 437. Valve assembly 440 includes housing 443, which surrounds an elongate portion of plunger 441. As shown in FIG. 16A, spring 442 biases plunger 441 toward the plug 404, such that the its distal end seats and seals on a projecting portion of wall 437, projection 409, such that fluid does not pass from proximal chamber 410a to distal chamber 410b. Contained within housing 443 can be a temperature expanded substance such as wax pellets that melt and expand at a predetermined temperature. As the pellets melt, the expansion cause plunger 441 to travel to the right, causing an opening around projection 409 for fluid to pass as is shown in FIG. 16B. Numerous alternative configurations can be used to move a sealing piston based on a temperature change, such as mechanisms incorporated bi-metallic springs or levers, one-way or two-way shaped memory alloys, and thermometer activated electronically driven actuators. Activation temperature can be chosen at a temperature proximate typical ablation temperature, e.g. approximate 50-70° C., more preferably 60-65° C. Alternatively, the activation temperature may be chosen at a temperature above 70° C., such as when valve assembly 440 is configured as an overload or failure protection assembly. In an alternative embodiment, initiation of cooling fluid delivery may be delayed, such as to allow a rapid increase in temperature of an electrode during initial energy delivery (e.g. no cooling fluid administered for an initial energy delivery time period), Once a particular electrode temperature is achieved, and/or a particular time period has elapsed, cooling fluid delivery is initiated.

Alternatively or additionally, fluid passing through valve assembly 440 may travel through a flow conduit that exits the side of the ablation catheter, such as a side wall of tip electrode 431, through shaft electrode 121, and/or through the wall of shaft 110. Alternatively or additionally, a pressure relief valve may be incorporated into the ablation catheter, not shown but preferably a spring activated piston valve which opens at a predetermined pressure. The output of the pressure relief valve may exit opening 401 of tip electrode 431, or it may exit at another catheter exit location. A pressure relief valve may be incorporated to open when cooling fluid is being delivered, and closed when no fluid is being delivered. Alternatively the pressure relief valve may be configured to open when an excessive pressure is reached, such as when a pressure is achieved that would damage one or more components of the ablation catheter or a pressure that would cause undesired trauma to the patient.

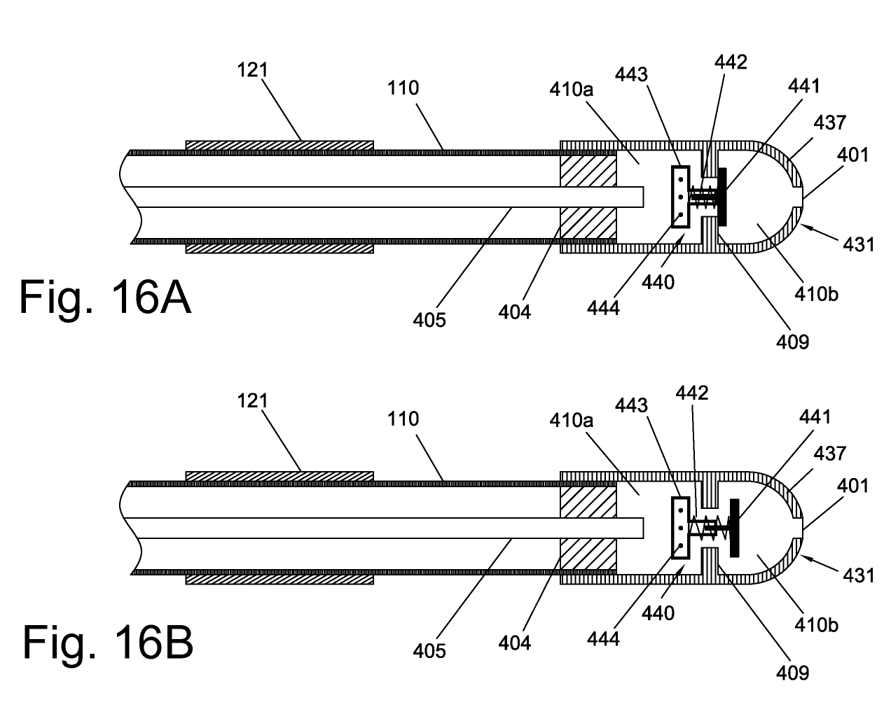

Referring now to FIG. 17, a side sectional view of the distal portion of an ablation catheter of the present invention is illustrated. Shaft 110 is preferably of the same construction and internal componentry as shaft 110 of FIG. 11. Shaft 110 includes multiple shaft electrodes 421 mounted to its outer diameter and tip electrode 431 mounted at its distal end, all electrodes preferably of platinum construction and all preferably including at least one temperature sensor such as a thermocouple. The distal end of tip electrode 431 includes walls 437 which define an exit hole 401.

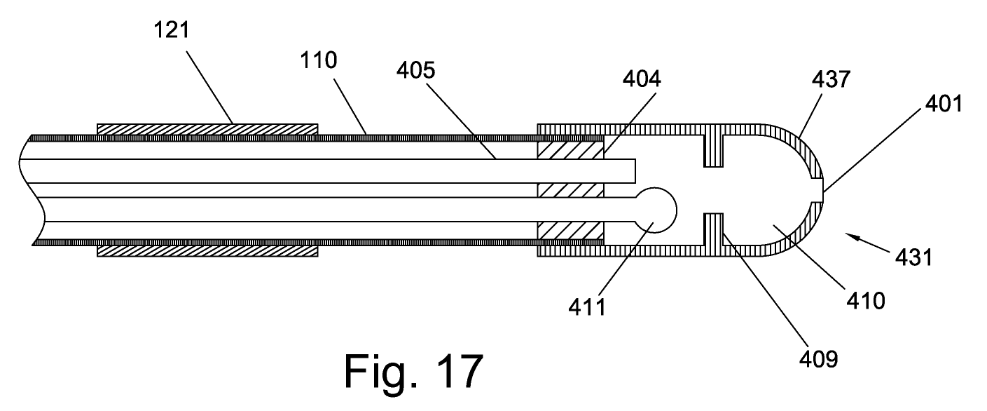

Fluid delivery tube 405 passes through sealing plug 404 and has a distal end 407 that terminates at a location flush with, just proximal to, or just distal to (as shown) plug 404's distal end, such that fluid enters chamber 410 at its proximal portion. Fluid is delivered to fluid delivery tube 405, as has been described in detail in reference to FIG. 5. Also passing through plug 404 is an elongate member that includes on its distal end, cooling member 411. Cooling member 411 functions as a heat sinking element to cool the fluid in chamber 410. Cooling member 411 may comprise one or more heat sinking surfaces in thermal communication with an heat transfer assembly located proximate the handle of the ablation catheter, handle and heat transfer assembly not shown. Cooling member 411 may receive a second cooling fluid, also from a device located proximate the handle of the ablation catheter, such as a refrigerant fluid which circulates through 411 without exiting into the patient's body, such as a refrigerant fluid which would be harmful to the body (i.e. not biocompatible). Cooling element 411 may include a semiconductor cooling device such as a Peltier device described in detail herebelow in reference to FIG. 18. Walls 437 of tip electrode 410 further define a projection 409, which is configured to cause the fluid exiting fluid delivery tube 405 to be in turbulent flow. The turbulent flow, as has been described hereabove, causes the hotter fluid along the inside of walls 437 to mix with the cooler fluid more distant from walls 437, resulting in an increased transfer of heat. If the fluid flowed very smoothly through the tip electrode 431, only the fluid actually touching walls 437 would absorb heat directly. The amount of heat transferred to the fluid from the walls 437 depends on the difference in temperature between the walls 437 and the fluid touching it. If the fluid that is in contact with the walls 437 heats up quickly, less heat will be transferred. By creating turbulence inside chamber 410, all of the fluid mixes together, reducing the temperature of the fluid touching the walls 437 so that more heat can be extracted, and all of the fluid inside chamber 410 is used effectively.

Referring now to FIG. 18, a side, partial cutaway view of the distal portion of an ablation catheter of the present invention is illustrated. A shaft, not shown but preferably of the same construction and internal componentry as shaft 110 of FIG. 11, attaches to the proximal end of tip electrode 131. Tip electrode 131 is preferably of platinum construction and preferably includes at least one temperature sensor such as a thermocouple. Walls 137 define a chamber 410, within which cooling element 461 resides. Cooling element 461 is a thermoelectric element such as Peltier Assembly which utilizes electrical energy, such as energy received through wires 462 from the RF generator or another electrical device of the ablation system of the present invention, to create a refrigeration effect.

Although the efficiency of a Peltier refrigerator is not high when compared to other refrigeration devices (typically only 5-10% efficient), the solid state circuitry of cooling element 461 can be manufactured very small and easily fit within the lumen of the ablation catheters as dimensioned hereabove. Simple electrical wires 462 travel proximally and attach to a standard DC energy source to create the cooling effect. Cooling element 461 is in good thermal contact with tip electrode 131 such as to efficiently absorb the heat generated during ablation. In an alternative embodiment, cooling fluid is also delivered through a thru-hole 463, with reciprocating fluid delivery, or through an exit hole in tip electrode 131, exit hole not shown.

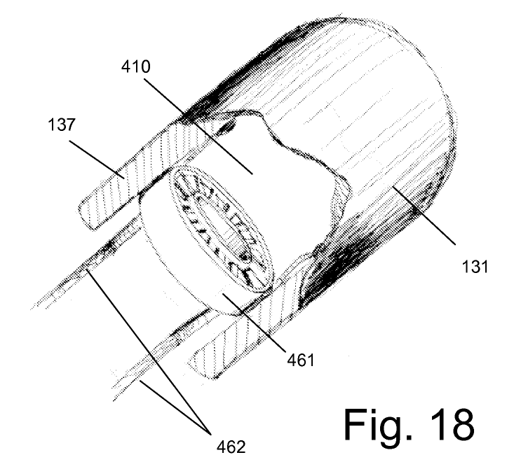

Referring now to FIGS. 19A and 19B, a side and end view, respectively, of a tip electrode assembly of the present invention is illustrated. Tip electrode 601a includes multiple elements, proximal electrode 604, distal electrode 603, and insulator 605 which is positioned to electrically isolate proximal electrode 604 and distal electrode 603. Insulator 605 is preferably constructed of a material selected from the group consisting of: plastic such as high temperature plastic such as polyimide; glass; rubber and other non-electrically conductive materials. Distal electrode 603, preferably constructed of platinum, is configured to deliver monopolar and/or bipolar energy to tissue when tip electrode assembly 601a is positioned orthogonal to tissue (e.g. distal electrode 603 is in contact with tissue and proximal electrode 604 is in circulating blood of a chamber of the heart). Proximal electrode 604, also preferably constructed of platinum, is configured to deliver monopolar and/or bipolar energy to tissue when tip electrode assembly 601a is positioned parallel to tissue (e.g. proximal electrode 604 is in contact with tissue and distal electrode 603 is in circulating blood of a chamber of the heart). In an alternative embodiment, bipolar energy can also be delivered between proximal electrode 604 and distal electrode 603.

Distal electrode 603 has a circular geometry with a diameter sized to create a specific tissue contact surface area. When electrode assembly 601a is placed with distal electrode 603 in contact with tissue (in the orthogonal position described above), a large portion of RF energy delivered by distal electrode 603 passes directly into tissue, with minimal energy passing through circulating blood or other non-target ablation areas. In a preferred embodiment, contact area between distal electrode 603 and tissue is 3-25 mm² (approximate electrode diameter 1.9-5.6 mm), more preferably 5-15 mm² (approximate electrode diameter 2.5-4.4 mm). The high efficiency design of electrode assembly 601a can effectively ablate tissue at lower power levels than standard, fully conductive tip electrodes. In an alternative embodiment, distal electrode 603 has a non-circular geometry.

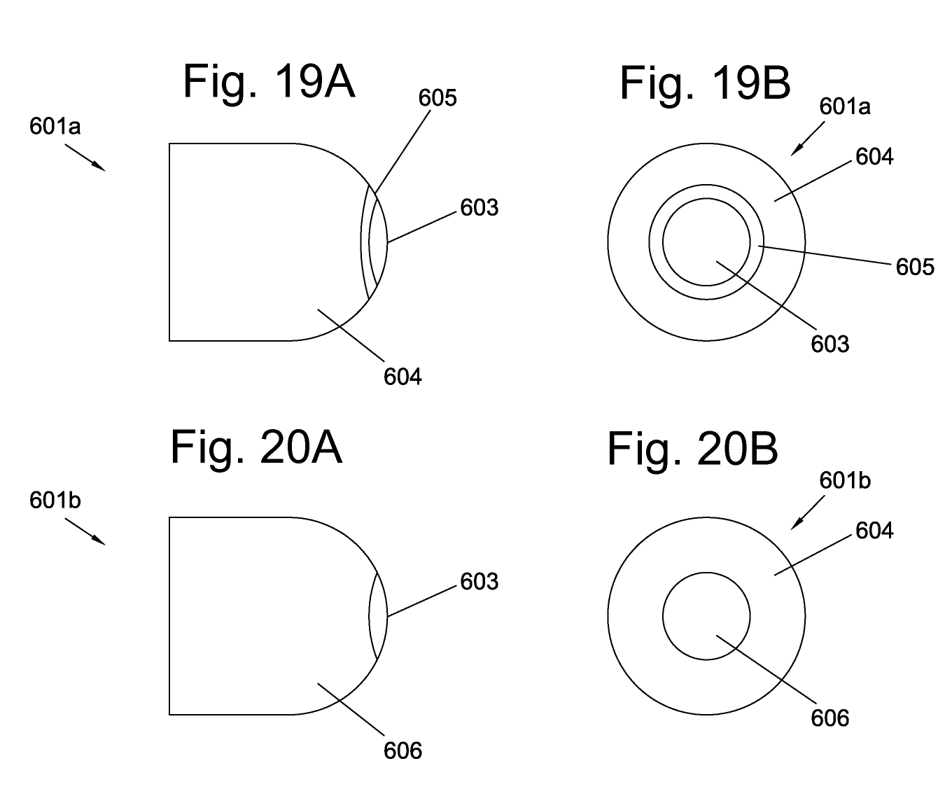

Referring now to FIGS. 20A and 20B, a side and end view, respectively, of a tip electrode assembly of the present invention is illustrated. Tip electrode 602a includes multiple elements, proximal insulator 606 and distal electrode 603. Insulator 606 is preferably constructed of a material selected from the group consisting of: plastic such as high temperature plastic such as polyimide; glass; rubber and other non-electrically conductive materials. Distal electrode 603, preferably constructed of platinum, is configured to deliver monopolar and/or bipolar energy to tissue when tip electrode assembly 602a is positioned orthogonal to tissue (e.g. distal electrode 603 is in contact with tissue and proximal insulator 605 is in circulating blood of a chamber of the heart).

Distal electrode 603 has a circular geometry with a diameter sized to create a specific tissue contact surface area. When electrode assembly 602a is placed with distal electrode 603 in contact with tissue (in the orthogonal position described above), a large portion of RF energy delivered by distal electrode 603 passes directly into tissue, with minimal energy passing through circulating blood or other non-target ablation areas. In a preferred embodiment, contact area between distal electrode 603 and tissue is 3-25 mm² (approximate electrode diameter 1.9-5.6 mm), more preferably 5-15 mm² (approximate electrode diameter 2.5-4.4 mm). The high efficiency design of electrode assembly 601a can effectively ablate tissue at lower power levels than standard, fully conductive tip electrodes. In an alternative embodiment, distal electrode 603 has a non-circular geometry.

Referring collectively to FIGS. 19A, 19B, 20A and 20B, the electrode assemblies each have a proximal end configured for attachment to a catheter shaft as has been described in detail hereabove, and are preferably constructed, in their energy delivery portions, of platinum. When attached to the catheter shaft, the tip electrodes may be configured to be in fluid communication with fluid delivery means provided by the catheter shaft. Each of these electrode assemblies are configured to be attached to a power delivery which receives energy from an RF energy generator. Each of these electrode assemblies include one or more thermocouples, as has been described in detail hereabove. The different portions of the electrodes may be attached to each other by one or more of: heat bonds; mechanical fastening means such as snaps and mating holes or dovetail joints; adhesives; and combinations of these.

In addition to the safe and efficient power delivery, the tip electrode assemblies of FIGS. 19A, 19B, 20A and 20B, a key advantage to the multiple portion design having a tip diameter that is larger than the electrode diameter. In applications such as treatment of a vessel of the heart such as treatment of the left atrium, small diameter devices have an increased risk of perforating through a wall of the heart, such as a perforation caused when an operator pushes the device into tissue during an ablation. The pressure exerted on tissue is inversely proportional to surface area of tissue contact, so these multi-portion designs allow an increased tissue contact area for the assembly, while avoiding unnecessary increase in electrode diameter. In a preferred embodiment, the assembly diameter is 4 mm or greater, and the electrode diameter is 3 mm or less. In other preferred embodiments, the radio of the electrode diameter is less than 80% of the tip diameter; less than 60% of the tip diameter: less than 50% of the tip diameter: or less than 25% of the tip diameter. Electrode diameters that are less than tip diameters provide numerous advantages including limiting the amount of energy that is delivered to non-target areas, such as circulating blood in a atrial ablation procedure.

It should be understood that numerous other configurations of the systems, devices and methods described herein can be employed without departing from the spirit or scope of this application. Numerous figures have illustrated typical dimensions, but it should be understood that other dimensions can be employed which result in similar functionality and performance.

It should be understood that the system includes multiple functional components, such as the RF generator and various ablation catheters of the present invention. A preferred ablation catheter consists of a catheter shaft, a shaft ablation assembly including at least one shaft ablation element, and a distal ablation assembly including at least one tip ablation element. Each of the catheters of the present invention may be introduced directly from the right atrium to the left atrium, or may pass through a previously placed transeptal sheath, such as a deflectable tip transeptal sheath. In a preferred system of the present invention, a transeptal sheath is included.

The cooling assemblies of the present invention may introduce fluid that is maintained within one or more blind lumens of the catheter, without entering the body of the patient. Preferably, the fluid passes through the catheter and exits at one or more of a tip electrode; a shaft electrode; and an exit hole in the shaft of the catheter. As has been described hereabove, the tip electrode may include a hollow chamber, such as a chamber in which cooling fluid circulates through, preferably by exiting an opening in the tip electrode. In an alternative or additional embodiment, one or more shaft electrodes may include a hollow chamber with any of the enhancements and modifications as have been described in reference to a chamber within a tip electrode.

The ablation catheters of the present invention include one or more ablation elements. In preferred embodiments, one or more ablation elements are electrodes configured to deliver RF energy. Other forms of energy, alternative or in addition to RF, may be delivered, including but not limited to: acoustic energy and ultrasound energy; electromagnetic energy such as electrical, magnetic, microwave and radiofrequency energies; thermal energy such as heat and cryogenic energies; chemical energy; light energy such as infrared and visible light energies; mechanical energy; radiation; and combinations thereof. The RF generator of the present invention may further provide one of the additional energy forms described immediately hereabove, in addition to the RF energy.

One or more ablation elements may comprise a drug delivery pump or a device to cause mechanical tissue damage such as a forwardly advancable spike or needle. The ablation elements can deliver energy individually, in combination with or in serial fashion with other ablation elements. The ablation elements can be electrically connected in parallel, in series, individually, or combinations thereof. The ablation catheter may include cooling means, such as fins or other heat sinking geometries, to prevent undesired tissue damage and/or blood clotting. The ablation elements may be constructed of various materials, such as plates of metal and coils of wire for RF energy delivery. The electrodes can take on various shapes including shapes used to focus energy such as a horn shape to focus sound energy, and shapes to assist in cooling such as a geometry providing large surface area. Wires and other flexible conduits are attached to the ablation elements, such as electrical energy carrying wires for RF electrodes or ultrasound crystals, and tubes for cryogenic delivery.

The ablation catheter of the present invention preferably includes a handle activating or otherwise controlling one or more functions of the ablation catheter. The handle may include various knobs or levers, such as rotating or sliding knobs which are operably connected to advancable conduits, or are operably connected to gear trains or cams which are connected to advancable conduits. These controls, such as knobs use to deflect a distal portion of a conduit, or to advance or retract the carrier assembly, preferably include a reversible locking mechanism such that a particular tip deflection or deployment amount can be maintained through various manipulations of the system.

The ablation catheter may include one or more sensors, such as sensors used to detect chemical activity; light; electrical activity; pH; temperature; pressure; fluid flow or another physiologic parameter. These sensors can be used to map electrical activity, measure temperature, or gather other information that may be used to modify the ablation procedure. In a preferred embodiment, one or more sensors, such as a mapping electrode, can also be used to ablate tissue.

Numerous components internal to the patient, such as the ablation elements, catheter shaft, shaft ablation assembly, distal ablation assembly, carrier arms or carrier assembly, may include one or more markers such as radiopaque markers visible under fluoroscopy, ultrasound markers, magnetic markers or other visual or other markers.

Selection of the tissue to be ablated may be based on a diagnosis of aberrant conduit or conduits, or based on anatomical location. RF energy may be delivered first, followed by another energy type in the same location, such as when a single electrode can deliver more than one type of energy, such as RF and ultrasound energy. Alternatively or additionally, a first procedure may be performed utilizing one type of energy, followed by a second procedure utilizing a different form of energy. The second procedure may be performed shortly after the first procedure, such as within four hours, or at a later date such as greater than twenty-four hours after the first procedure. Numerous types of tissue can be ablated utilizing the devices, systems and methods of the present invention. For example, the various aspects of the invention have application in procedures for ablating tissue in the prostrate, brain, gall bladder, uterus, other organs and regions of the body, and a tumor, preferably regions with an accessible wall or flat tissue surface. In the preferred embodiment, heart tissue is ablated, such as left atrial tissue.

In another preferred embodiment of the system of the present invention, an ablation catheter and a heat sensing technology are included. The heat sensing technology, includes sensor means that may be placed on the chest of the patient, the esophagus or another area in close enough proximity to the tissue being ablated to directly measure temperature effects of the ablation, such as via a temperature sensor, or indirectly such as through the use of an infrared camera. In these embodiments, the RFG includes means of receiving the temperature information from the heat sensing technology, similar to the handling of the temperature information from thermocouples of the ablation catheters. This additional temperature information can be used in one or more algorithms for power delivery, as has been described above, and particularly as a safety threshold which shuts off or otherwise decreased power delivery. A temperature threshold will depend on the location of the heat sensing technology sensor means, as well as where the ablation energy is being delivered. The threshold may be adjustable, and may be automatically configured.

Numerous kit configurations are also to be considered within the scope of this application. An ablation catheter is provided with one or more tip electrodes, one or more shaft electrodes and a shaft with a deflectable distal portion, such as an asymmetrically deflectable distal portion.

Though the ablation device has been described in terms of its preferred endocardial and percutaneous method of use, the ablation elements may be used on the heart during open heart surgery, open chest surgery, or minimally invasive thoracic surgery. Thus, during open chest surgery, a short catheter or cannula carrying the ablation elements may be inserted into the heart, such as through the left atrial appendage or an incision in the atrium wall, to apply the ablation elements to the tissue to be ablated. Also, the ablation elements may be applied to the epicardial surface of the atrium or other areas of the heart to detect and/or ablate arrhythmogenic foci from outside the heart.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims. In addition, where this application has listed the steps of a method or procedure in a specific order, it may be possible, or even expedient in certain circumstances, to change the order in which some steps are performed, and it is intended that the particular steps of the method or procedure claim set forth herebelow not be construed as being order-specific unless such order specificity is expressly stated in the claim.

We claim:

1. An ablation catheter for performing a medical procedure on a patient, said catheter comprising:
an elongate shaft having a lumen therein, said shaft having proximal and distal ends;
a tip ablation electrode mounted on the distal end and a plug, the tip electrode having walls that, together with the plug, define a chamber, the tip electrode further having a fluid exit port;
a cooling fluid delivery system, said cooling fluid delivery system comprising a connection to a fluid source and a fluid delivery tube within the lumen of the elongate shaft, said fluid delivery tube penetrating said plug; and
a valve assembly in located in the chamber between the fluid delivery tube and the fluid exit port, said valve assembly movable between an open and closed position, said valve preventing the flow of cooling fluid through the fluid delivery tube when in the closed position.

2. The ablation catheter of claim 1, wherein said valve assembly has a biasing mechanism.

3. The ablation catheter of claim 2, wherein said biasing mechanism biases the valve assembly in the closed position.

4. The ablation catheter of claim 1, wherein said valve assembly further comprises a housing, said housing containing a temperature responsive device, said temperature responsive material configured to cause said valve assembly to move to the open position at a predetermined temperature.

5. The ablation catheter of claim 4, wherein said temperature responsive material is elected from the group consisting of bi-metallic springs, shaped memory alloys, and thermometer-activated electronically-driven actuators.

6. The ablation catheter of claim 4, wherein said temperature responsive material is a temperature-expandable substance that melts and expands at a predetermined temperature, such moving the valve assembly from the closed position to the open position.

7. An ablation system for ablating cardiac tissue, the system comprising:
a source of cooling fluid;
an RF generator for generating energy having an RF frequency; and
a catheter including:
an elongate, tubular shaft having proximal and distal ends, and a fluid delivery lumen extending with the shaft substantially between the proximal and distal ends, the fluid delivery lumen being in fluid communication with the source of cooling fluid;
a shaft ablation electrode mounted on the shaft near the distal end thereof;
a tip ablation electrode mounted on the distal end of the shaft, the tip ablation electrode defining a fluid exit port in fluid communication with the fluid delivery lumen;
a valve assembly located within the catheter along the fluid delivery lumen adjacent the tip electrode, the valve assembly being movable between an open position and a closed position;
conductors for transmitting RF energy to the shaft ablation electrode and tip electrode;
a processor having:
means for setting RF energy parameters for the delivery of RF energy to the ablation electrodes, the parameters including the time duration to deliver RF energy to the ablation electrodes, an RF energy ON time at which delivery of RF energy is initiated and an RF energy OFF time at which delivery of RF energy is stopped; and
means for setting valve parameters for the operation of the valve assembly, the valve parameters including a valve opening time at which the valve assembly is moved to the open position, the valve opening time being set at a predetermined offset relative the RF energy ON time, and a valve closing time at which the valve assembly is moved to the closed position.

8. The ablation system of claim 7 in which the means for setting valve parameters for the operation of the valve assembly includes means for determining the valve closing time as an offset based on the time duration of delivery of RF energy.

9. The ablation system of claim 7 in which the means for setting valve parameters for the operation of the valve assembly include means for determining the valve closing time as an offset relative to the RF energy OFF time.

10. A method of ablating cardiac tissue comprising:
providing a catheter comprising:
an elongate shaft having a lumen therein, said shaft having proximal and distal ends;
a tip ablation electrode mounted on the distal end of the shaft, the tip electrode having a fluid exit port;
shaft ablation electrodes mounted on said shaft proximal to said tip electrode;
a cooling fluid delivery system, said cooling fluid delivery system comprising a fluid source and a fluid delivery tube within the lumen of the elongate shaft;
a valve assembly located between the fluid delivery tube and the fluid exit port, said valve assembly movable between an open and closed position;
wherein said shaft ablation electrode and said tip electrode have conductors for receiving RF energy;
determining parameters for the delivery of RF energy to said ablation elements, said parameters including a target ablation temperature;
initiating delivery of RF energy;
measuring the ablation temperature; and
moving the valve assembly between the open and closed position to maintain the ablation temperature at the target ablation temperature.

11. The method of claim 10, wherein said ablation catheter has a plurality of shaft ablation electrodes.

12. The method of claim 10, wherein the step of delivering ablation energy comprises delivering energy selected from the group consisting of bipolar energy and unipolar energy.

13. The method of claim 10, wherein the step of delivering ablation energy comprises delivering a combination of unipolar and bipolar energy.

14. A method of operating an ablation catheter comprising:
providing a catheter comprising:
an elongate shaft having a lumen therein, said shaft having proximal and distal ends;
a tip ablation electrode mounted on the distal end of the shaft, and a plug, the tip ablation electrode having walls that, together with the plug, define a chamber, the tip electrode further having a fluid exit port;
a shaft ablation electrode mounted on the shaft proximal to the tip electrode;
a cooling fluid delivery system, said cooling fluid delivery system comprising a fluid source and a fluid delivery tube within the lumen of the elongate shaft;
a valve assembly located between the fluid delivery tube and the fluid exit port, said valve assembly movable between an open and closed position;
wherein said shaft ablation electrode and said tip electrode have conductors for receiving RF ablation energy;
setting parameters for the delivery of RF ablation energy to said ablation electrodes, said parameters including the time duration to deliver RF energy to the ablation elements;
setting parameters for the operation of the valve assembly, said parameters including a valve opening offset time, said valve opening offset time indicating the amount of time before or after the initiation of RF ablation energy delivery to open said valve assembly, and a valve closing offset time, said valve closing offset time indicating the amount of time before or after the initiation of RF ablation energy delivery to open said valve assembly;
initiating delivery of RF ablation energy;
opening said valve assembly according to the valve opening offset time;
terminating delivery of RF ablation energy; and
closing said valve assembly according to the valve closing offset time.
